(19) United States
(12) Patent Application Publication
(10) Pub. No.: US 2026/0091016 A1
(43) Pub. Date: Apr. 2, 2026

(54) Composition for preventing and/or combating neuroinflammation, particularly in neuropsychiatric and neurological diseases

(71) Applicant: BEE TERAPI, PARIS (FR)

(72) Inventors: Jérémie LAVAUR, PARIS (FR); Nicolas CARDINAULT, Belleneuve (FR)

(21) Appl. No.: 18/904,297

(22) Filed: Oct. 2, 2024

(51) Int. Cl. A61K 31/352; A61K 31/216; A61K 35/644; A61P 25/28

(52) U.S. Cl. CPC A61K 31/352; A61K 31/216; A61K 35/644; A61P 25/28

(57) ABSTRACT

The present invention relates to a composition comprising a mixture of molecules, said mixture comprising at least chrysin, pinocembrin and CAPE, for its use in the prevention or treatment of neuroinflammation and/or diseases or disorders associated with neuroinflammation.

### TECHNICAL FIELD

The present invention relates to the technical field of preventing and/or combating neuroinflammation and/or diseases or disorders associated with neuroinflammation. In particular, the invention concerns a composition comprising a mixture of specific molecules for preventing and/or combating neuroinflammation and/or diseases or disorders associated with neuroinflammation.

### PRIOR ART

Neuroinflammation is a complex immune mechanism designed to protect the brain from aggression. To protect itself, the brain has immunocompetent cells, known as microglial cells or microglia. Microglia play a central role in maintaining homeostasis and mediating inflammation in the brain.

Indeed, microglia constitute the first level of defense against pathogens, stress episodes or any disturbance of the cerebral environment.

A stimulus can lead to a gradual transformation of microglial morphology, from a highly branched cell in contact with astrocytes in a quiescent state to a phagocytic cell of the macrophage type during severe aggression of the central nervous system (CNS).

Thus, microglia can exist in different states: a resting state, which is relatively inactive but can perform surveillance functions, or in one of two functionally distinct activation states, M1 and M2.

The M1 state is induced by a signal such as IFN-gamma or lipopolysaccharide (LPS), and responds by releasing inflammatory cytokines such as TNF-alpha, IL-1 beta and reactive oxygen species/reactive nitrogen species (ROS/RNS).

The M2 state has an anti-inflammatory effect, blocking the release of pro-inflammatory cytokines, ingesting debris, promoting tissue repair, and releasing neurotrophic factors.

Astrocytes are also important regulators of immunity in the CNS.

The whole of the microglial and astrocytic response through the proliferation of immune cells and the release of cytotoxic and/or inflammatory factors is grouped together under the term “neuroinflammation”.

Neuroinflammation is an inflammatory state of the CNS involved in a large number of situations, notably during normal aging, depression, cognitive disorders, anxiolytic disorders, but also in certain neurological diseases such as Alzheimer's disease (AD), Parkinson's disease (PD), multiple sclerosis (MS), sleep apnea, and amyotrophic lateral sclerosis (ALS).

To date, there is no standard treatment for neuroinflammation. As a result, manufacturers are always on the lookout for a new alternative treatment.

There is therefore a need for a new alternative treatment aimed at preventing and/or combating neuroinflammation and/or neuroinflammation-related diseases or disorders.

### SUMMARY OF THE INVENTION

To meet this need, the invention proposes a new composition (a) comprising a mixture of molecules including pinocembrin, chrysin, and caffeic acid phenethyl ester (CAPE).

Surprisingly, the inventors have discovered that the combination of 3 specific polyphenols, namely pinocembrin, chrysin and CAPE, had a synergistic effect in preventing and/or combating neuroinflammation and/or diseases or disorders associated with neuroinflammation.

Preferably, composition (a) according to the invention is liquid and contains no alcohol. According to a preferred embodiment, composition (a) according to the invention is a liquid, anhydrous, alcohol-free composition. Thus, composition (a) according to this embodiment has an oily appearance.

Advantageously, composition (a) according to the invention has a significant effect on neuroinflammation while avoiding the undesirable effects of alcohol.

Composition (a) according to the invention may comprise a mixture of molecules of natural or synthetic origin.

According to one embodiment, composition (a) according to the invention comprises a mixture of molecules of natural origin, in particular obtained from a natural raw material, preferably from propolis.

Preferentially, composition (a) according to the invention comprises a mixture of molecules of natural origin, characterized in that said mixture is a propolis extract.

Preferably, composition (a) according to the invention comprises a mixture of molecules of natural origin, characterized in that said mixture is a propolis extract comprising at least 70% of polyphenols in aglycone form, relative to the total polyphenols of the extract.

Of particular interest is the presence of a majority of polyphenols in aglycone form in composition (a) according to the invention. Aglycone polyphenols are more easily absorbed by enterocytes and therefore better assimilated by the body, particularly in humans and animals.

More particularly, when composition (a) comprises a propolis extract, the latter is obtainable by a method comprising the implementation of the following steps:
a) Grinding at least one raw propolis
b) Macerating the propolis powder obtained in step a) in an extraction solvent
c) Filtering the macerate obtained in step b) and recovering the filtrate
d) Evaporating the filtrate obtained in step c) forming a residue
e) Mixing the residue obtained in step d) with a mixture of organic solvent and aqueous solvent with stirring
f) Settling the mixture obtained in step e) and recovering the organic phase
g) Washing the organic phase obtained in step f) with anhydrous sodium sulfate salts
h) Filtering the organic phase obtained in step g)
i) Evaporating the organic solvent and recovering of a propolis extract selectively enriched with pinocembrin, chrysin and CAPE.

Finally, according to a last aspect, the invention relates to composition (a) according to the invention for its use in preventing and/or combating neuroinflammation and/or diseases or disorders associated with neuroinflammation.

Further features and advantages will become apparent from the detailed description of the invention, examples, and figures, which are illustrative only and by no means limit the scope of the invention.

### BRIEF DESCRIPTION OF THE FIGURES

FIG. 1A is a graphical representation showing the effect on IFN gamma expression, dose response, of composition (a) according to the invention comprising a mixture of molecules of synthetic origin on primary human microglial cultures in the presence or absence of LPS. FIG. 1B is a graphical representation showing the effect on TNF-alpha expression, dose response, of composition (a) according to the invention comprising a mixture of molecules of synthetic origin on primary human microglial cultures in the presence or absence of LPS. FIG. 1C is a graphical representation showing the effect on IL-6 expression, dose response, of composition (a) according to the invention comprising a mixture of molecules of synthetic origin on primary human microglial cultures in the presence or absence of LPS. FIG. 1D is a graphical representation showing the effect on IL-1 beta expression, dose response, of composition (a) according to the invention comprising a mixture of molecules of synthetic origin on primary human microglial cultures in the presence or absence of LPS. FIG. 1E is a graphical representation showing the effect on IL 18 expression, dose response, of composition (a) according to the invention comprising a mixture of molecules of synthetic origin on primary human microglial cultures in the presence or absence of LPS. FIG. 1F is a graphical representation showing the effect on IL-10 expression, dose response, of composition (a) according to the invention comprising a mixture of molecules of synthetic origin on primary human microglial cultures in the presence or absence of LPS.

FIG. 2a is a graphical representation showing the effect on IFN-Gamma expression, dose-response, of composition (a) according to the invention comprising a mixture of molecules of synthetic origin on primary human microglial cultures in the presence or absence of LPS.

FIG. 2b is a graphical representation showing the effect on TNF-alpha expression, dose-response, of composition (a) according to the invention comprising a mixture of molecules of synthetic origin on primary human microglial cultures in the presence or absence of LPS.

FIG. 2c is a graphical representation showing the effect on IL-6 expression, dose-response, of composition (a) according to the invention comprising a mixture of molecules of synthetic origin on primary human microglial cultures in the presence or absence of LPS.

FIG. 2d is a graphical representation showing the effect on IL-1 beta expression, dose-response, of composition (a) according to the invention comprising a mixture of molecules of synthetic origin on primary human microglial cultures in the presence or absence of LPS.

FIG. 2e is a graphical representation showing the effect on IL-18 expression, dose-response, of composition (a) according to the invention comprising a mixture of molecules of synthetic origin on primary human microglial cultures in the presence or absence of LPS.

FIG. 2f is a graphical representation showing the effect on IL-10 expression, dose-response, of composition (a) according to the invention comprising a mixture of molecules of synthetic origin on primary human microglial cultures in the presence or absence of LPS.

FIG. 3 is a graphical representation depicting the anxiolytic effect of composition (a) according to the invention comprising propolis extract in the elevated plus maze behavioral test.

FIG. 4 is a graphical representation depicting the dose-response inhibitory effect of composition (b) according to the invention comprising propolis extract on the enzymatic activity of monoamine oxidase A.

FIG. 5 is a graphical representation depicting the dose-response inhibitory effect of composition (b) according to the invention comprising a mixture of molecules of synthetic origin on the enzymatic activity of monoamine oxidase A.

FIG. 6 is a graphical representation depicting the dose-response inhibitory effect of composition (b) according to the invention comprising propolis extract on the enzymatic activity of monoamine oxidase B.

FIG. 7 is a graphical representation depicting the dose-response inhibitory effect of composition (b) according to the invention comprising a mixture of molecules of synthetic origin on the enzymatic activity of monoamine oxidase B.

FIG. 8 is a graphical representation depicting the dose-response inhibitory effect of composition (c) according to the invention comprising propolis extract on the enzymatic activity of acetylcholinesterase.

FIG. 9 is a graphical representation depicting the dose-response inhibitory effect of the composition according to the invention comprising a mixture of molecules of synthetic origin on the enzymatic activity of acetylcholinesterase.

FIG. 10 is a graphical representation depicting the neuroprotective and antioxidant dose-response effect of composition (a) according to the invention comprising propolis extract on dopaminergic neurons treated with neurotoxins (mpp+ and 6-OHDA).

FIG. 11 is a graphical representation depicting the anxiolytic effect of composition (a) according to the invention in the elevated plus maze behavioral test via time spent in open arms.

FIG. 12 is a graphical representation depicting the anxiolytic effect of composition (a) according to the invention in the elevated plus maze behavioral test via distance traveled in maze arms.

FIG. 13 is a graphical representation depicting the sedative effect of composition (a) according to the invention in the Open-Field test via distance traveled.

FIG. 14 is a graphical representation of the quantitative analyses of ACTH stress hormone in different groups of treated mice.

### DETAILED DESCRIPTION OF THE INVENTION

### Definition

“Propolis”, in the sense of the invention, is a resinous, gummy, balsamic substance made from the wax and salivary secretions of bees, mixed with substances harvested from the bark and buds of plants and trees.

“Raw propolis” in the sense of the invention is propolis obtained directly from the beehive, without any prior processing step.

“Propolis extract” in the sense of the invention is an extract comprising at least one set of molecules, the extract being obtained from any harvested propolis converted by an extraction method enabling the impurities present in the raw extract to be removed and/or the propolis to be concentrated into one or more of its constituents.

“Alcohol-free” or “non-alcoholic” in the sense of the invention means that the composition according to the invention contains less than 0.1% alcohol. This alcohol content can be measured using techniques well known to the person skilled in the art, such as distillation, electronic densitometry or ebulliometry.

“Cold grinding” in the sense of the invention means that the raw propolis has been exposed to a temperature below 0° C. for a period of one hour prior to grinding according to the invention.

“Systemic absorption” in the sense of the invention means the ability of polyphenols to cross the enterocyte membrane and be measured in plasma.

“Aglycone form” in the sense of the invention means that the polyphenols present in the extract are no longer bound to sugars and/or fatty acids.

“Mild saponification” in the sense of the invention means chemical hydrolysis carried out with a strong base concentration of between 0.01 and 0.1 M, enabling the extract according to the invention to be concentrated in polyphenols in aglycone form without degrading them.

“Natural raw material” in the sense of the invention means a raw product found in nature.

“Combat” or “treat” in the sense of the invention means to reduce the pathological state, that is, a reduction in the progression of the pathology, stabilization, reversal or regression, or even an interruption or inhibition of the progression of the pathology.

“Synthetic origin” in the sense of the invention means that the molecule is obtained by chemical synthesis.

### Composition (a)

In response to the need for a new treatment to prevent and/or combat neuroinflammation and/or diseases or disorders associated with neuroinflammation, the inventors have developed a composition (a) comprising a mixture of molecules including pinocembrin, chrysin and CAPE.

Advantageously, the composition according to the invention comprises the synergistic combination of 3 polyphenols (pinocembrin, chrysin and CAPE) to prevent and/or combat neuroinflammation and/or diseases or disorders associated with neuroinflammation.

Preferably, composition (a) according to the invention comprises a mixture of molecules with a CAPE/pinocembrin/chrysin ratio of between 1/2/2 and 1/3/3.

According to one variant, composition (a) according to the invention may also comprise at least one additive.

Composition (a) according to the invention may contain as an additive at least one compound chosen from oils, waxes, surfactants, co-surfactants, thickeners and/or gelling agents, humectants, colorants, preservatives, fillers, firming agents, sequestrants, anti-caking agents and mixtures thereof, without this list being limiting.

Of course, the person skilled in the art will take care to choose any additional, active or non-active compounds, and their quantity, such that the advantageous properties of composition (a) according to the invention are not, or substantially not, altered by the envisaged additive.

Composition (a) according to the invention comprises a mixture of molecules which may be of synthetic or natural origin.

When composition (a) according to the invention comprises a mixture of molecules of synthetic origin, said mixture of molecules preferentially comprises:
at least 5 μM pinocembrin;
at least 4.3 μM chrysin; and
at least 1.76 μM CAPE.

Advantageously, such concentrations are sufficient to generate a synergistic effect on neuroinflammation.

According to a particularly preferred embodiment, composition (a) according to the invention comprises or consists of a propolis extract. According to another embodiment, composition (a) according to the invention consists essentially of a propolis extract.

Preferentially, composition (a) according to the invention comprises a mixture of molecules of natural origin, characterized in that said mixture is a propolis extract.

Advantageously, according to this embodiment, composition (a) according to the invention offers a natural alternative treatment for preventing and/or combating neuroinflammation and/or diseases or disorders associated with neuroinflammation.

Preferentially, composition (a) according to the invention comprises a mixture of molecules of natural origin, characterized in that said mixture is a propolis extract comprising pinocembrin, chrysin, and CAPE.

Surprisingly, the inventors have succeeded in obtaining a composition (a) according to the invention comprising a concentrated propolis extract selectively enriched with polyphenols of interest, namely pinocembrin, chrysin and CAPE.

When composition (a) comprises a propolis extract, this may come from any well-identified botanical origin. Preferentially, said propolis extract is obtained from at least one propolis selected from poplar propolis, Baccharis propolis and Dalbergia propolis and mixtures thereof.

According to a particularly preferred embodiment, said propolis extract is obtained from poplar propolis.

Preferably, composition (a) according to the invention is in liquid form at room temperature, preferably at 22° C.

In a preferred embodiment, composition (a) according to the invention comprises a mixture of molecules of natural origin, characterized in that said mixture is a propolis extract having a viscosity of between 25 and 900 mPa·s, preferably between 50 and 450 mPa·s.

Composition (a) according to the invention can also have a viscosity of between 25 and 900 mPa·s, preferentially between 50 and 450 mPa·s, particularly when composition (a) comprises or consists of a propolis extract.

In the context of the invention, viscosity is measured at 20° C. using a Brookfield viscometer well known in the prior art.

Advantageously, the viscosity of propolis extract enables it to be easily incorporated into composition (a) according to the invention.

According to one embodiment, when composition (a) comprises a propolis extract, this comprises by mass of total polyphenols of the extract:
at least 2.6% CAPE;
at least 8% pinocembrin; and
at least 8% chrysin.

According to another embodiment, composition (a) according to the invention comprises a mixture of molecules also including at least one molecule chosen from apigenin, galangin and combinations thereof.

Thus, composition (a) according to the invention may comprise a mixture of molecules comprising pinocembrin, chrysin, CAPE, apigenin and galangin.

Advantageously, when composition (a) according to the invention comprises the 5 above-mentioned polyphenols, said composition (a) exhibits a multifactorial effect. Indeed, when composition (a) according to the invention comprises the combination of pinocembrin, chrysin, CAPE, apigenin and galangin, it can prevent and/or combat not only neuroinflammation, but also other pathologies, notably depression, anxiety, Parkinson's disease, and memory disorders. More particularly, according to this embodiment, composition (a) may induce a neuroprotective effect.

According to another embodiment, composition (a) according to the invention comprises a mixture of molecules of natural origin, characterized in that said mixture is a propolis extract comprising at least 18% total polyphenols relative to the total mass of the extract, preferentially at least 19%, even more preferentially at least 20%.

Preferentially, said propolis extract comprises a content of between 18 and 35% of total polyphenols relative to the total mass of the composition, even more preferentially between 19 and 35%, especially between 20 and 30%.

The content of total polyphenols is preferentially measured by the reference method described in the following document “Bankova, Vassya & Popova, Milena & Trusheva, Boryana. (2016). New emerging fields of application of propolis. Macedonian Journal of Chemistry and Chemical Engineering. 35. 1 10.20450/mjcce.2016.864”.

In a particularly preferred embodiment, composition (a) according to the invention comprises a mixture of molecules of natural origin, characterized in that said mixture is a propolis extract comprising, by weight of total polyphenols of the extract:
at least 0.4% apigenin, preferentially at least 0.6% apigenin
at least 2.6% CAPE, preferentially at least 2.9%
at least 6.0% galangin, preferentially at least 7%
at least 8.0% chrysin, preferentially at least 8.5% and
at least 8.0% pinocembrin, preferentially at least 9.2%.

Thus, composition (a) according to the invention may comprise a propolis extract, said extract being concentrated in polyphenols of major interest in human health, in particular pinocembrin, galangin, apigenin, chrysin and CAPE and mixtures thereof.

Preferably, composition (a) according to the invention comprises a mixture of molecules of natural origin, characterized in that said mixture is a propolis extract comprising at least 70% of polyphenols in aglycone form, relative to the total polyphenols of the extract, preferentially at least 85%, even more preferentially at least 90%.

The concentration of aglycone polyphenols in the extract according to the invention can be measured by comparing an analysis of the extract before and after treatment with any enzyme capable of hydrolyzing β-glucosidic bonds, preferentially glucosidases.

Of particular interest is the presence of a majority of polyphenols in aglycone form in propolis extract. Aglycone polyphenols are more easily absorbed by enterocytes and therefore better assimilated by the body, particularly in humans and animals.

According to a variant, when composition (a) comprises a propolis extract, said extract also comprises, by mass of the total polyphenols of the extract:
a caffeic acid content of less than 0.02%
a ferulic acid content of less than 0.02%
a p-Coumaric acid content of less than 0.08%.

According to one embodiment, composition (a) and/or the propolis extract do not comprise at least one molecule chosen from farnesyl, caffeic acid, ferulic acid and p-Coumaric acid.

According to another embodiment, composition (a) and/or propolis extract do not comprise at least two molecules selected from farnesyl, caffeic acid, ferulic acid and p-Coumaric acid.

According to another embodiment, composition (a) and/or propolis extract do not comprise farnesyl, caffeic acid, ferulic acid, p-Coumaric acid and farnesyl.

Advantageously, when composition (a) according to the invention comprises a propolis extract, the propolis extract is selectively concentrated in at least 3, preferably 5 polyphenols and has a very low, or even zero, content of other molecules initially present in the raw propolis extract.

When composition (a) according to the invention comprises a propolis extract, said extract may be obtained by any suitable method. According to a particularly preferred embodiment, composition (a) according to the invention comprises a mixture of molecules of natural origin, characterized in that said mixture is a propolis extract obtainable according to a method comprising the following steps:
a) Grinding at least one raw propolis
b) Macerating the propolis powder obtained in step a) in an extraction solvent
c) Filtering the macerate obtained in step b) and recovering the filtrate
d) Evaporating the filtrate obtained in step c) forming a residue
e) Mixing the residue obtained in step d) with a mixture of organic solvent and aqueous solvent with stirring
f) Settling the mixture obtained in step e) and recovering the organic phase
g) Washing the organic phase obtained in step f) with anhydrous sodium sulfate salts
h) Filtering the organic phase obtained in step g)
i) Evaporating the organic solvent and recovering a liquid propolis extract comprising chrysin, CAPE and pinocembrin.

Preferentially, said method for obtaining a propolis extract further comprises a step h′) for selecting the molecules of interest, in particular chrysin, CAPE, and pinocembrin.

In the context of the invention, the extraction solvent of step b) is a mixture of organic solvent, aqueous solvent, and a strong base. For the purposes of the invention, step b) is a saponification step, preferentially a mild saponification step.

Advantageously, the strong base will break the sugar-polyphenol bonds, thus enabling the majority of polyphenols to be obtained in aglycone form, making them easily assimilable by the intestinal mucosa, in particular directly assimilable by the intestinal mucosa without prior enzymatic treatment.

According to another embodiment of the invention, composition (a) according to the invention comprises a mixture of molecules of natural origin, characterized in that said mixture is a propolis extract obtained by a method comprising a propolis saponification step, preferentially a mild saponification step.

When composition (a) comprises a mixture of molecules of synthetic origin, the mixture of molecules may be obtained by any suitable method.

According to one embodiment, composition (a) according to the invention comprises a mixture of molecules of synthetic origin obtainable according to a method comprising the following steps:
a) Selecting molecules comprising:
   at least CAPE, pinocembrin, and chrysin; and
   optionally, at least one molecule chosen from galangin and apigenin.
b) Mixing the molecules selected in step a) in a suitable solvent.

According to one embodiment, said suitable solvent is alcohol or a hydroalcoholic solution.

### Method for Obtaining Composition (a) Comprising a Mixture of Molecules Of Synthetic Origin

In order to obtain composition (a) according to the invention comprising a mixture of molecules of synthetic origin, the inventors have developed an obtaining method comprising at least the implementation of the following steps:
a) Selecting molecules of interest comprising:
   at least CAPE, pinocembrin, and chrysin; and
   optionally, at least one molecule chosen from galangin and apigenin.
b) Mixing the molecules selected in step a) in a suitable solvent.

According to one embodiment, said suitable solvent in step b) is alcohol or a hydroalcoholic solution.

### Composition (b)

According to another aspect, the invention concerns a composition (b) comprising a mixture of molecules comprising galangin and apigenin for preventing and/or combating depression.

Advantageously, composition (b) according to the invention comprises the synergistic combination of 2 polyphenols (galangin and apigenin) to prevent and/or combat depression.

Preferably, composition (b) according to the invention comprises a mixture of molecules with a galangin/apigenin ratio of between 11/1 and 15/1.

Preferentially, composition (b) according to the invention is liquid and contains no alcohol. According to a preferred embodiment, composition (b) according to the invention is a liquid, anhydrous, alcohol-free composition.

Thus, composition (b) according to this embodiment has an oily appearance.

According to one variant, composition (b) according to the invention may also comprise at least one additive.

Composition (b) according to the invention may contain as an additive at least one compound chosen from oils, waxes, surfactants, co-surfactants, thickeners and/or gelling agents, humectants, colorants, preservatives, fillers, firming agents, sequestrants, anti-caking agents and mixtures thereof, without this list being limiting.

Of course, the person skilled in the art will take care to choose any additional, active or non-active compounds, and their quantity, such that the advantageous properties of composition (b) according to the invention are not, or substantially not, altered by the envisaged additive.

Composition (b) according to the invention comprises a mixture of molecules which may be of synthetic or natural origin.

When composition (b) according to the invention comprises a mixture of molecules of synthetic origin, said mixture of molecules preferentially comprises:
at least 15 nM apigenin; and
at least 140 nM galangin.

Advantageously, such concentrations are sufficient to generate a synergistic effect on monoamine oxidase A involved in depression.

According to one embodiment, composition (b) according to the invention comprises a mixture of molecules of natural origin, in particular obtained from a natural raw material, preferentially from propolis.

Preferentially, composition (b) according to the invention comprises a mixture of molecules of natural origin, characterized in that said mixture is a propolis extract.

According to a particularly preferred embodiment, composition (b) according to the invention comprises or consists of a propolis extract. According to another embodiment, composition (b) according to the invention consists essentially of a propolis extract.

Advantageously, according to this embodiment, the composition according to the invention offers a natural alternative treatment for preventing and/or combating depression.

When composition (b) according to the invention comprises a propolis extract, said extract may be a propolis extract as defined in any of the embodiments previously described for composition (a).

Preferentially, composition (b) according to the invention comprises a mixture of molecules of natural origin, characterized in that said mixture is a propolis extract comprising galangin and apigenin.

Surprisingly, the inventors have succeeded in obtaining a composition according to the invention comprising a propolis extract selectively enriched with polyphenols of interest, namely galangin and apigenin.

Preferably, composition (b) according to the invention is in liquid form at room temperature, preferentially at 22° C.

In a preferred embodiment, composition (b) according to the invention comprises a mixture of molecules of natural origin, characterized in that said mixture is a propolis extract having a viscosity of between 25 and 900 mPa·s, preferentially between 50 and 450 mPa·s.

Composition (b) according to the invention can also have a viscosity of between 25 and 900 mPa·s, preferentially between 50 and 450 mPa·s, particularly when composition (b) comprises or consists of a propolis extract.

Advantageously, composition (b) according to the invention does not precipitate on contact with water, in particular the water present in the oral cavity, thereby enabling intestinal absorption of polyphenols.

In one embodiment, composition (b) according to the invention comprises a mixture of molecules of natural origin, characterized in that said mixture is a propolis extract comprising, by weight of total polyphenols of the extract:
at least 0.4% apigenin, preferentially at least 0.6% apigenin; and
at least 6% galangin, preferentially at least 7%.

According to another embodiment, composition (b) according to the invention comprises a mixture of molecules also including at least one molecule chosen from CAPE, pinocembrin, chrysin, and combinations thereof.

According to one variant, composition (b) according to the invention comprises a mixture of molecules of natural origin, characterized in that said mixture is a propolis extract further comprising, by weight of total polyphenols of the extract:
a caffeic acid content of less than 0.02%
a ferulic acid content of less than 0.02%
a p-Coumaric acid content of less than 0.08%.

According to one embodiment, composition (b) and/or the propolis extract do not comprise at least one molecule chosen from farnesyl, caffeic acid, ferulic acid and p-Coumaric acid.

According to another embodiment, composition (b) and/or propolis extract do not comprise at least two molecules selected from farnesyl, caffeic acid, ferulic acid and p-Coumaric acid.

According to another embodiment, composition (b) and/or propolis extract do not comprise farnesyl, caffeic acid, ferulic acid, p-Coumaric acid and farnesyl.

Advantageously, when composition (b) according to the invention comprises a propolis extract, the propolis extract is selectively concentrated in at least 2 polyphenols (apigenin and galangin) and has a very low, or even zero, content of other molecules initially present in the raw propolis extract.

When composition (b) according to the invention comprises a propolis extract, said extract may be obtained by any suitable method. According to a particularly preferred embodiment, composition (b) according to the invention comprises a mixture of molecules of natural origin, characterized in that said mixture is a propolis extract obtainable according to a method comprising the following steps:
a) Grinding at least one raw propolis
b) Macerating the propolis powder obtained in step a) in an extraction solvent
c) Filtering the macerate obtained in step b) and recovering the filtrate
d) Evaporating the filtrate obtained in step c) forming a residue
e) Mixing the residue obtained in step d) with a mixture of organic solvent and aqueous solvent with stirring
f) Settling the mixture obtained in step e) and recovering the organic phase
g) Washing the organic phase obtained in step f) with anhydrous sodium sulfate salts
h) Filtering the organic phase obtained in step g)
i) Evaporating the organic solvent and recovering a liquid propolis extract comprising apigenin and galangin.

Preferentially, said method for obtaining a propolis extract further comprises a step h′) for selecting the molecules of interest, in particular apigenin and galangin.

In the context of the invention, the extraction solvent of step b) is a mixture of organic solvent, aqueous solvent, and a strong base. For the purposes of the invention, step b) is a saponification step, preferentially a mild saponification step.

According to another embodiment of the invention, composition (b) according to the invention comprises a mixture of molecules of natural origin, characterized in that said mixture is a propolis extract obtained by a method comprising a propolis saponification step, preferentially a mild saponification step.

When composition (b) comprises a mixture of molecules of synthetic origin, the mixture of molecules may be obtained by any suitable method.

According to one embodiment, composition (b) according to the invention comprises a mixture of molecules of synthetic origin obtainable according to a method comprising the following steps:
a) Selecting molecules of interest comprising:
   at least apigenin and galangin; and
   optionally, at least one molecule chosen from pinocembrin, CAPE and chrysin.
b) Mixing the molecules selected in step a) in a suitable solvent.

### Method for Obtaining Composition (b) Comprising a Mixture of Molecules Of Synthetic Origin

In order to obtain composition (b) according to the invention comprising a mixture of molecules of synthetic origin, the inventors have developed an obtaining method comprising at least the implementation of the following steps:
a) Selecting molecules of interest comprising:
   at least apigenin and galangin; and
   optionally, at least one molecule chosen from pinocembrin, CAPE and chrysin.
b) Mixing the molecules selected in step a) in a suitable solvent.

According to one embodiment, said suitable solvent in step b) is alcohol or a hydroalcoholic solution.

### Composition (c)

According to another aspect, the invention concerns a composition (c) comprising a mixture of molecules comprising galangin and CAPE for preventing and/or combating Alzheimer's disease.

Advantageously, composition (c) according to the invention comprises the synergistic combination of 2 polyphenols (galangin and CAPE) to prevent and/or combat Alzheimer's disease.

Preferably, composition (c) according to the invention comprises a mixture of molecules with a galangin/CAPE ratio of between 2/1 and 3/1.

According to one variant, composition (c) according to the invention may also comprise at least one additive.

Composition (c) according to the invention may contain as an additive at least one compound chosen from oils, waxes, surfactants, co-surfactants, thickeners and/or gelling agents, humectants, colorants, preservatives, fillers, firming agents, sequestrants, anti-caking agents and mixtures thereof, without this list being limiting.

Of course, the person skilled in the art will take care to choose any additional, active or non-active compounds, and their quantity, such that the advantageous properties of composition (c) according to the invention are not, or substantially not, altered by the envisaged additive.

Preferentially, composition (c) according to the invention is liquid and contains no alcohol. According to a preferred embodiment, composition (c) according to the invention is a liquid, anhydrous, alcohol-free composition.

Thus, composition (c) according to this embodiment has an oily appearance.

Composition (c) according to the invention comprises a mixture of molecules which may be of synthetic or natural origin.

When composition (c) according to the invention comprises a mixture of molecules of synthetic origin, said mixture of molecules preferentially comprises:
at least 1.41 μM galangin; and
at least 0.73 μM CAPE.

Advantageously, such concentrations are sufficient to generate a synergistic effect on acetylcholinesterase involved in Alzheimer's disease.

According to one embodiment, composition (c) according to the invention comprises a mixture of molecules of natural origin, in particular obtained from a natural raw material, preferably from propolis.

Preferentially, composition (c) according to the invention comprises a mixture of molecules of natural origin, characterized in that said mixture is a propolis extract.

According to a particularly preferred embodiment, composition (c) according to the invention comprises or consists of a propolis extract. According to another embodiment, composition (c) according to the invention consists essentially of a propolis extract.

Advantageously, according to this embodiment, composition (c) according to the invention offers a natural alternative treatment for preventing and/or combating Alzheimer's disease.

When composition (c) according to the invention comprises a propolis extract, the propolis extract may be a propolis extract as defined in any of the embodiments previously described for composition (a) or (b).

Preferentially, composition (c) according to the invention comprises a mixture of molecules of natural origin, characterized in that said mixture is a propolis extract comprising galangin and CAPE.

Surprisingly, the inventors have succeeded in obtaining a composition (c) according to the invention comprising a propolis extract selectively enriched with polyphenols of interest, namely galangin and CAPE.

Preferably, composition (c) according to the invention is in liquid form at room temperature, preferentially at 22° C.

In a preferred embodiment, composition (c) according to the invention comprises a propolis extract with a viscosity of between 25 and 900 mPa·s, preferentially between 50 and 450 mPa·s.

Composition (c) according to the invention can also have a viscosity of between 25 and 900 mPa·s, preferentially between 50 and 450 mPa·s, particularly when composition (c) comprises or consists of a propolis extract.

Advantageously, composition (c) according to the invention does not precipitate on contact with water, in particular the water present in the oral cavity, thereby enabling intestinal absorption of polyphenols.

In one embodiment, composition (c) according to the invention comprises a mixture of molecules of natural origin, characterized in that said mixture is a propolis extract comprising, by weight of total polyphenols of the extract:
at least 2.6% CAPE, preferentially at least 2.9%; and
at least 6% galangin, preferentially at least 7%.

According to another embodiment, composition (c) according to the invention comprises a mixture of molecules also including at least one molecule chosen from apigenin, pinocembrin, chrysin, and combinations thereof.

According to one variant, composition (c) according to the invention comprises a mixture of molecules of natural origin, characterized in that said mixture is a propolis extract further comprising, by weight of total polyphenols of the extract:
a caffeic acid content of less than 0.02%
a ferulic acid content of less than 0.02%
a p-Coumaric acid content of less than 0.08%.

According to one embodiment, composition (c) and/or the propolis extract do not comprise at least one molecule chosen from farnesyl, caffeic acid, ferulic acid and p-Coumaric acid.

According to another embodiment, composition (c) and/or propolis extract do not comprise at least two molecules selected from farnesyl, caffeic acid, ferulic acid and p-Coumaric acid.

According to another embodiment, composition (c) and/or propolis extract do not comprise farnesyl, caffeic acid, ferulic acid, p-Coumaric acid and farnesyl.

Advantageously, when composition (c) according to the invention comprises a propolis extract, the propolis extract is selectively concentrated in at least 2 polyphenols (galangin and CAPE) and has a very low, or even zero, content of other molecules initially present in the raw propolis extract.

When composition (c) according to the invention comprises a propolis extract, said extract may be obtained by any suitable method. According to a particularly preferred embodiment, composition (c) according to the invention comprises a mixture of molecules of natural origin, characterized in that said mixture is a propolis extract obtainable according to a method comprising the following steps:
a) Grinding at least one raw propolis
b) Macerating the propolis powder obtained in step a) in an extraction solvent
c) Filtering the macerate obtained in step b) and recovering the filtrate
d) Evaporating the filtrate obtained in step c) forming a residue
e) Mixing the residue obtained in step d) with a mixture of organic solvent and aqueous solvent with stirring
f) Settling the mixture obtained in step e) and recovering the organic phase
g) Washing the organic phase obtained in step f) with anhydrous sodium sulfate salts
h) Filtering the organic phase obtained in step g)
i) Evaporating the organic solvent and recovering a liquid propolis extract comprising CAPE and galangin.

Preferentially, said method for obtaining a propolis extract further comprises a step h′) for selecting the molecules of interest, in particular CAPE and galangin.

In the context of the invention, the extraction solvent of step b) is a mixture of organic solvent, aqueous solvent, and a strong base. For the purposes of the invention, step b) is a saponification step, preferentially a mild saponification step.

When composition (c) comprises a mixture of molecules of synthetic origin, the mixture of molecules may be obtained by any suitable method.

According to one embodiment, composition (c) according to the invention comprises a mixture of molecules of synthetic origin obtainable according to a method comprising the following steps:
a) Selecting molecules of interest comprising:
   at least CAPE and galangin; and
   optionally, at least one molecule chosen from pinocembrin, apigenin and chrysin and combinations thereof.
b) Mixing the molecules selected in step a) in a suitable solvent.

### Method for Obtaining Composition (c) Comprising a Mixture of Molecules Of Synthetic Origin

In order to obtain composition (c) according to the invention comprising a mixture of synthetic molecules, the inventors have developed an obtaining method comprising at least the implementation of the following steps:
a) Selecting molecules of interest comprising:
   at least CAPE and galangin; and
   optionally, at least one molecule chosen from pinocembrin, apigenin and chrysin and combinations thereof.
b) Mixing the molecules selected in step a) in a suitable solvent.

According to one embodiment, said suitable solvent in step b) is alcohol or a hydroalcoholic solution.

Method for Obtaining Composition (a), (b) or (c) Comprising a Mixture Of Molecules of Natural Origin, Characterized in that Said Mixture is a Propolis Extract

According to another aspect and in order to obtain composition (a), (b) or (c) according to the invention comprising a mixture of molecules of natural origin, characterized in that said mixture is a propolis extract, the inventors have developed a method for obtaining a propolis extract comprising at least the implementation of the following steps:
a) Grinding at least one raw propolis,
b) Macerating the propolis powder obtained in step a) in an extraction solvent,
c) Filtering the macerate obtained in step b) and recovering the filtrate,
d) Evaporating the filtrate obtained in step c) forming a residue,
e) Mixing the residue obtained in step d) with a mixture of organic solvent and aqueous solvent with stirring,
f) Settling the mixture obtained in step e) and recovering the organic phase,
g) Washing the organic phase obtained in step f) with anhydrous sodium sulfate salts,
h) Filtering the organic phase obtained in step g),
i) Evaporating the organic solvent and recovering a propolis extract.

Preferably, the raw propolis used in step a) comprises at least 45% resin, preferentially at least 55% resin, even more preferentially at least 60% resin

Advantageously, the resin concentration of raw propolis is a reliable indicator of its quality, the higher the resin content of raw propolis, the higher the concentration of active ingredients in the extract according to the invention.

The raw propolis used in step a) can come from any well-identified botanical origin. Preferentially, raw propolis is chosen from poplar propolis, Baccharis propolis, Dalbergia propolis and mixtures thereof.

Advantageously, poplar propolis, Baccharis propolis and Dalbergia propolis are naturally rich in polyphenols.

Preferably, the raw propolis from step a) is poplar propolis.

The grinding in step a) can be carried out by crushing, percussion, abrasion, or shearing. Preferentially, the grinding in step a) is carried out by shearing.

According to a preferred embodiment, the grinding in step a) is carried out cold, that is, at a temperature below 0° C.

In a particularly preferred embodiment, the particles constituting the ground matter obtained in step a) have a diameter of between 0.1 and 1 mm, preferentially between 0.2 and 0.6 mm.

Advantageously, the grinding step according to the invention greatly improves the polyphenol concentration of said extract by increasing the exchange surface between the raw propolis and the extraction solvent of step b).

In the context of the invention, the extraction solvent of step b) comprises an organic solvent, an aqueous solvent, and a strong base. For the purposes of the invention, step b) is a saponification step, preferentially a mild saponification step.

Advantageously, the strong base will break the sugar-polyphenol bonds, enabling the majority of polyphenols to be obtained in aglycone form, making them easily assimilated by the intestinal mucosa.

The aglycone form of the polyphenols in propolis extract enables them to be directly assimilated by the mucosa without prior enzymatic treatment.

Preferably, the extraction solvent in step b) comprises a mixture of organic solvent/aqueous solvent and a strong base, the solvent/strong base ratio being between 1/10 and ¼ by volume. The organic solvent/aqueous solvent mixture of step b) preferentially has a ratio of between 40/60 and 90/10 by volume, even more preferentially a ratio of between 70/30 and 85/15 by volume.

The organic solvent present in the extraction solvent of step b) is preferentially chosen from ethanol and methanol.

The aqueous solvent present in the extraction solvent of step b) can be water.

Preferably, the strong base of step b) is present at a concentration of between 0.01 and 0.1 M, preferentially between 0.02 and 0.08 M, even more preferably at a concentration of 0.03 M.

Advantageously, a strong base concentration of between 0.01 and 0.1 M enables the propolis extract to concentrate polyphenols in aglycone form without degrading them.

The strong base present in the extraction solvent in step b) is preferentially chosen from potassium hydroxide and sodium hydroxide.

According to a particularly preferred embodiment, the extraction solvent in step b) is sodium hydroxide.

The extraction solvent in step b) is preferentially chosen from a mixture of ethanol/water and sodium hydroxide or methanol/water and sodium hydroxide.

The maceration in step b) is carried out with continuous agitation, preferentially using a vortex adapted to the volume of ground matter, a paddle mixer or a sonication or ultrasonic system.

The maceration time in step b) can vary between 2 hours and 10 days, depending on the volume of ground matter from step a).

Filtration in step c) can be carried out by successive filtration, centrifugal filtration, or successive gravity settling with filter filtration or plate filtration.

According to a preferred embodiment, the filtration in step c) comprises successive centrifugal filtration with nylon filters ranging from 200 μm down to 5 μm.

Advantageously, the filtrate obtained in step c) is clear and sediment free.

The evaporation in step d) or i) can be carried out under vacuum at a pressure between 100 and 300 mbar and at a temperature between 3° and 60° C.

Evaporation in step d) is preferentially carried out under vacuum at a pressure of between 100 and 300 mbar and a temperature of between 3° and 60° C., even more preferentially at a pressure of 140 mbar and a temperature of between 45 and 50° C.

In the context of the invention, the evaporation must be rapid, while keeping the temperature as low as possible to preserve the polyphenols in the propolis.

Advantageously, the evaporation in step d) produces a residue with an alcohol content of less than 0.1%. Techniques for determining alcohol content are well known to the person skilled in the art. In the context of the invention, content is determined by distillation and by electron densitometry or by ebulliometry.

Preferably, the mixing in step e) is carried out with stirring using a vortex adapted to the volume of residue or a paddle mixer.

In a particularly preferred embodiment of the invention, the mixing in step e) is carried out with stirring until the residue obtained in step d) is completely dissolved.

The organic solvent used in step e) is preferentially selected from ethyl acetate, methyl acetate and dimethyl ether and mixtures thereof.

The aqueous solvent in step e) can be water.

Preferably, the organic solvent/aqueous solvent mixture in step e) is an ethyl acetate/water mixture.

The organic solvent/aqueous solvent mixture of step e) preferentially has a ratio of between 30/70 and 90/10 by volume, even more preferentially a ratio of between 50/50 and 80/20 by volume.

In a particularly preferred embodiment of the invention, the organic solvent/aqueous solvent mixture of step e) is an ethyl acetate/water mixture in a 50/50 volume ratio.

The residue/solvent ratio of the mixture from step e) is preferentially between ⅓ and ¼ (weight/volume).

The settling in step f) can be carried out by gravity settling or centrifugation.

Step g) of washing the organic phase with sodium sulfate salts advantageously traps any remaining water molecules in the organic phase.

Step h) of filtering the organic phase obtained in step g) removes salt crystals from the organic phase, thus obtaining a water-free organic phase.

The filtration in step h) is carried out on a filter with sufficiently narrow mesh to remove salt crystals from the organic phase. The person skilled in the art is perfectly capable of choosing a filter with a mesh size adapted to the size of the salt crystals. As a non-limiting example, Whatman grade 4 paper or a coffee filter is sufficient to remove said crystals.

Preferentially, said method for obtaining a propolis extract further comprises a step h′) for selecting the molecules of interest.

The evaporation in step i) is preferentially carried out under vacuum at a pressure of between 150 and 300 mbar and a temperature of between 3° and 60° C., even more preferentially at a pressure of between 200 and 250 mbar and a temperature of between 4° and 45° C.

Advantageously, the method according to the invention makes it possible to obtain a viscous fluid propolis extract that is not very sticky at room temperature and comprises a high content of polyphenols of interest.

More particularly, the method according to the invention makes it possible to obtain a composition (a), (b) or (c) comprising a liquid alcohol-free propolis extract with a viscosity of between 20 and 450 mPa·s.

The method according to the invention makes it possible to selectively concentrate the content of polyphenols of interest.

### Composition (a) for Use

The object of the present invention is therefore a composition (a) comprising a mixture of molecules comprising pinocembrin, chrysin and CAPE for preventing and/or combating neuroinflammation and/or diseases or disorders associated with neuroinflammation.

Particularly advantageously, the inventors have developed a composition (a) comprising a mixture of specific molecules comprising the combination of 3 polyphenols (CAPE, pinocembrin and chrysin) with a synergistic effect on neuroinflammation.

In a preferred embodiment, the composition (a) comprises a mixture of molecules consisting essentially of pinocembrin, chrysin and CAPE for preventing and/or combating neuroinflammation and/or diseases or disorders associated with neuroinflammation.

Advantageously, these 3 polyphenols are of major interest in human health, particularly neurology.

Advantageously, thanks to the central role of neuroinflammation, composition (a) according to the invention can prevent and/or combat a large number of CNS disorders, and more particularly diseases or disorders associated with neuroinflammation.

Diseases or disorders associated with neuroinflammation particularly include the following pathologies:
Neurological pathologies selected from Alzheimer's disease (and related Lewy body diseases, Huntington's disease, frontotemporal dementia, cerebrovascular disease/vascular dementia), vascular lesions, stroke lesions, cerebral ischemia, Parkinson's (and related diseases such as multisystem atrophy, progressive supranuclear palsy, Lewy body dementia, essential tremor), Epilepsy, Amyotrophic Lateral Sclerosis (ALS), autoimmune diseases such as Multiple Sclerosis (MS), narcolepsy, SARS-CoV2
Neuro-psychiatric pathologies chosen from schizophrenia, bipolar disorder, anxiety disorders, depression, panic attacks, post-traumatic shock, autism
The other pathologies involved are chosen from:
   neuroviral or neurobacterial infections such as neuroinflammation associated with acute infectious meningitis, infectious encephalitis, brain abscesses, neuro-COVID-19 infections, HIV/AIDS.
   glioblastomas, such as the neuroinflammation associated with brain tumors and cancer-related cognitive disorders
   diabetes
   pain
   age-related macular degeneration (AMD) and glaucoma
   irritable bowel syndrome
   osteoporosis
   hypertension
   cardiovascular disorders
   perioperative neurocognitive disorders
   sleep apnea syndrome

Preferentially, composition (a) according to the invention can be used to prevent and/or combat Alzheimer's disease, Parkinson's disease, multiple system atrophy, Huntington's disease, posterior cortical atrophy, Pick's disease, epilepsy, vascular dementia, frontotemporal dementia, Lewy body dementia, amyotrophic lateral sclerosis, Korsakoff syndrome, alcohol withdrawal, ischemia, neonatal ischemia, head trauma, stroke, vascular dementia, cerebral ischemia, multiple system atrophy, progressive supranuclear palsy, amyotrophic lateral sclerosis, multiple sclerosis, narcolepsy, SARSCoV-2, schizophrenia, bipolar disorder, anxiety disorders, depressive disorders, panic attacks, post-traumatic stress disorder, and autism.

Advantageously, it is the combination of CAPE, chrysin and pinocembrin that makes it possible to obtain an effect on all the above-mentioned pathologies.

According to one embodiment, composition (a) according to the invention can be used to prevent or treat anxiety.

Advantageously, composition (a) exerts a non-GABA-A receptor-dependent action. Indeed, to date, 30% of patients are insensitive to antidepressant treatments targeting GABA-A receptors. There is therefore a need for a new treatment that can exert a non-GABA-A receptor-dependent action on anxiety and/or depression.

According to one embodiment, composition (a) can be used to prevent neuroinflammation and/or diseases or disorders associated with neuroinflammation, preferentially to induce a neuroprotective effect on neurons.

Preferably, composition (a) according to the invention can be used to reduce oxidative stress or apoptosis in neuronal cells.

In this way, composition (a) is able to induce a neuroprotective effect, thus preventing the onset of neuroinflammation.

Advantageously, composition (a) according to the invention can simultaneously prevent and combat neuroinflammation by inducing neuroprotection of neurons while reducing the inflammatory state of the CNS.

According to a particular embodiment, composition (a) according to the invention can be used to decrease the expression of at least one, in particular at least two, preferentially all cytokines selected from IFN-gamma, TNF-alpha, IL-6, IL-1 beta and IL-18 in the CNS.

Preferentially, the extract according to the invention can be used to decrease the expression of IFN-gamma and TNF-alpha.

Advantageously, a reduction in the concentration of at least one, in particular at least two, preferentially all, of the aforementioned cytokines helps prevent and/or combat neuroinflammation.

When composition (a) according to the invention comprises a mixture of molecules comprising CAPE, pinocembrin, galangin, chrysin and apigenin, said composition can be used as an inhibitor:
monoamine oxidase A; and/or
monoamine oxidase B; and/or
acetylcholinesterase.

Monoamine oxidase A is an enzyme involved in depression. Inhibition of this enzyme can be used to treat certain depressive disorders.

Monoamine oxidase B is an enzyme that notably degrades dopamine in the CNS. Monoamine oxidase B inhibitors are used in particular to treat Parkinson's disease.

Thus, according to this preferred embodiment, composition (a) according to the invention can be used to prevent and/or combat Parkinson's disease.

Acetylcholinesterase is an enzyme that regulates the concentration of acetylcholine by degrading it to prevent excessive action. Acetylcholine is a molecule involved in the transmission of information between neurons, and in the process of memorizing information. In Alzheimer's disease, patients experience a constant loss of acetylcholine.

By inhibiting acetylcholinesterase, acetylcholinesterase inhibitors correct acetylcholine deficiency.

Preferably, composition (a) according to the invention can thus be used to prevent and/or combat Alzheimer's disease.

In one variant, composition (a) according to the invention can be used as a drug or dietary supplement for humans or animals.

Advantageously, when composition (a) comprises a propolis extract, it causes few or no side effects when used in humans or animals.

### Composition (b) for Use

Another subject of the present invention is a composition (b) comprising a mixture of molecules comprising apigenin and galangin for preventing and/or combating depression.

Particularly advantageously, the inventors have developed a composition (b) comprising a mixture of molecules including the combination of 2 polyphenols (apigenin and galangin) with a synergistic effect on depression, in particular on monoamine oxidase A and/or B.

In a preferred embodiment, composition (b) comprises a mixture of molecules consisting essentially of apigenin and galangin to prevent and/or combat depression.

Advantageously, these 2 polyphenols are of major interest in human health, particularly neurology.

Surprisingly, the inventors have discovered the synergistic combination of these 2 polyphenols to prevent and/or combat depression.

More particularly, composition (b) can be used as a monoamine oxidase A inhibitor.

Monoamine oxidase A is an enzyme involved in depression. Inhibition of this enzyme can be used to treat certain depressive disorders.

According to another embodiment, composition (b) according to the invention can be used as a monoamine oxidase B inhibitor.

Monoamine oxidase B is an enzyme that notably degrades dopamine in the CNS. Monoamine oxidase B inhibitors are used in particular to treat Parkinson's disease.

Thus, according to a particularly preferred embodiment, composition (b) according to the invention can be used to prevent and/or combat Parkinson's disease.

In one variant, composition (b) according to the invention can be used as a drug or dietary supplement for humans or animals.

Advantageously, when composition (b) comprises a propolis extract, it causes few or no side effects when used in humans or animals.

### Composition (c) for Use

Finally, the present invention also relates to a composition (c) comprising a mixture of molecules comprising galangin and CAPE for preventing and/or combating Alzheimer's disease.

Particularly advantageously, the inventors have developed a composition (c) comprising a mixture of molecules including the combination of 2 polyphenols (CAPE and galangin) with a synergistic effect on Alzheimer's disease, in particular on acetylcholinesterase.

In a preferred embodiment, composition (c) comprises a mixture of molecules consisting essentially of galangin and CAPE for preventing and/or combating Alzheimer's disease.

Advantageously, these 2 polyphenols are of major interest in human health, particularly neurology.

Surprisingly, the inventors have discovered the synergistic combination of these two polyphenols to prevent and/or combat Alzheimer's disease.

More particularly, composition (c) can be used as an inhibitor of acetylcholinesterase.

Acetylcholinesterase is an enzyme that regulates the concentration of acetylcholine by spurring its destruction it to prevent excessive action. Acetylcholine is a molecule involved in the transmission of information between neurons, and in the process of memorizing information. In Alzheimer's disease, patients experience a constant loss of acetylcholine.

By inhibiting acetylcholinesterase, inhibitors of this enzyme correct acetylcholine deficiency.

In another embodiment, composition (c) can be used to preserve memory.

In one variant, composition (c) according to the invention can be used as a drug or dietary supplement for humans or animals.

Advantageously, when composition (c) comprises a propolis extract, it causes few or no side effects when used in humans or animals.

### EXAMPLES

### Example 1: Example of a Method for Obtaining Composition (a) Comprising a Propolis Extract

The method for obtaining composition (a) according to the invention comprising a propolis extract comprises carrying out the following steps:
a) Grinding previously frozen raw poplar propolis to obtain particles with a diameter of between 0.2 and 0.6 mm
b) Macerating the propolis powder obtained in step a) in a mixture of ethanol/water in a 70/30 volume ratio and sodium hydroxide at a concentration of 0.025 M
c) Filtering the macerate obtained in step b) by successive filtration and recovering the filtrate
d) Vacuum evaporation at a pressure of 140 mbar and a temperature of 45° C. of the filtrate obtained in step c) forming a residue containing an alcohol content of less than 0.1%
e) Mixing the residue obtained in step d) with a mixture of ethyl acetate and water with stirring
f) Settling the mixture obtained in step e) by gravity settling and recovering the ethyl acetate phase
g) Washing the organic phase obtained in step f) with anhydrous sodium sulfate salts
h) Filtering the organic phase obtained in step g)
i) Vacuum-evaporating ethyl acetate at a pressure of 200 mbar and a temperature of 45° C. and recovering a liquid propolis extract comprising chrysin, CAPE and pinocembrin.

Said method makes it possible to obtain an alcohol-free liquid propolis extract selectively concentrated with polyphenols of interest. Said extract comprises a significant content of at least 3 polyphenols whose concentrations measured using the reference High Performance Liquid Chromatography (HPLC) method as described by Bankova, V. et al. 2016 are presented in Table 1.

Table 1: Average concentration of polyphenols of interest obtained from 10 extracts using the method described above.

TABLE 1

| | CAPE | pinocembrin | chrysin |
|---|---|---|---|
| Concentration | 0.52 | 2.0 | 2.48 |
| (g/100 g) | | | |

### Example 2: Example of a Method for Obtaining Composition (b) Comprisinq a Propolis Extract

The method for obtaining composition (b) according to the invention comprising a propolis extract comprises carrying out the following steps:
a) Grinding previously frozen raw poplar propolis to obtain particles with a diameter of between 0.2 and 0.6 mm
b) Macerating the propolis powder obtained in step a) in a mixture of ethanol/water in a 70/30 volume ratio and sodium hydroxide at a concentration of 0.025 M
c) Filtering the macerate obtained in step b) by successive filtration and recovering the filtrate
d) Vacuum evaporation at a pressure of 140 mbar and a temperature of 45° C. of the filtrate obtained in step c) forming a residue containing an alcohol content of less than 0.1%
e) Mixing the residue obtained in step d) with a mixture of ethyl acetate and water with stirring
f) Settling the mixture obtained in step e) by gravity settling and recovering the ethyl acetate phase
g) Washing the organic phase obtained in step f) with anhydrous sodium sulfate salts
h) Filtering the organic phase obtained in step g)
i) Vacuum-evaporating ethyl acetate at a pressure of 200 mbar and a temperature of 45° C. and recovering a liquid propolis extract comprising apigenin and galangin.

Said method makes it possible to obtain an alcohol-free liquid propolis extract selectively concentrated with polyphenols of interest. Said extract comprises a significant content of at least 2 polyphenols whose concentrations measured using the reference High Performance Liquid Chromatography (HPLC) method as described by Bankova, V. et al. 2016 are presented in Table 2.

Table 2: Average concentration of polyphenols of interest obtained from 10 extracts using the method described above.

TABLE 2

| | Apigenin | galangin |
|---|---|---|
| Concentration | 0.12 | 1.52 |
| (g/100 g) | | |

### Example 3: Example of a Method for Obtaining Composition (c) Comprising a Propolis Extract

The method for obtaining composition (c) according to the invention comprising a propolis extract comprises carrying out the following steps:
a) Grinding previously frozen raw poplar propolis to obtain particles with a diameter of between 0.2 and 0.6 mm
b) Macerating the propolis powder obtained in step a) in a mixture of ethanol/water in a 70/30 volume ratio and sodium hydroxide at a concentration of 0.025 M
c) Filtering the macerate obtained in step b) by successive filtration and recovering the filtrate
d) Vacuum evaporation at a pressure of 140 mbar and a temperature of 45° C. of the filtrate obtained in step c) forming a residue containing an alcohol content of less than 0.1%
e) Mixing the residue obtained in step d) with a mixture of ethyl acetate and water with stirring
f) Settling the mixture obtained in step e) by gravity settling and recovering the ethyl acetate phase
g) Washing the organic phase obtained in step f) with anhydrous sodium sulfate salts
h) Filtering the organic phase obtained in step g)
i) Vacuum-evaporating ethyl acetate at a pressure of 200 mbar and a temperature of 45° C. and recovering a liquid propolis extract comprising CAPE and galangin.

Said method makes it possible to obtain an alcohol-free liquid propolis extract selectively concentrated with polyphenols of interest. Said extract comprises a significant content of at least 2 polyphenols whose concentrations measured using the reference High Performance Liquid Chromatography (HPLC) method as described by Bankova, V. et al. 2016 are presented in Table 3.

Table 3: Average concentration of polyphenols of interest obtained from 10 extracts using the method described above.

TABLE 3

| | CAPE | galangin |
|---|---|---|
| Concentration | 0.52 | 1.52 |
| (g/100 g) | | |

### Example 4: Example of a Method for Obtaining Composition (a) According to the Invention

The method for obtaining composition (a) according to the invention comprising a mixture of synthetic molecules comprises carrying out the following steps:
a) Selecting the molecules: pinocembrin, chrysin and CAPE
b) Mixing the molecules selected in step a) in a hydroalcoholic solution to obtain a composition (a) comprising:
   at least 5 μM pinocembrin;
   at least 4.3 μM chrysin; and
   at least 1.76 μM CAPE.

### Example 5: Example of a Method for Obtaining Composition (b) According to the Invention

The method for obtaining composition (b) according to the invention comprising a mixture of synthetic molecules comprises carrying out the following steps:
a) Selecting the molecules, namely apigenin and galangin
b) Mixing the molecules selected in step a) in a hydroalcoholic solution to obtain a composition (b) comprising:
   between 15 and 20 nM apigenin, in particular 18 nM; and
   between 120 and 200 nM galangin, notably 140 nM.

### Example 6: Example of a Method for Obtaining Composition (c) According to The Invention

The method for obtaining composition (c) according to the invention comprising a mixture of synthetic molecules comprises carrying out the following steps:
a) Selecting the molecules, namely apigenin and galangin
b) Mixing the molecules selected in step a) in a hydroalcoholic solution to obtain a composition (c) comprising:
   at least 1.41 μM galangin; and
   at least 0.73 μM CAPE.

### Example 7: Measuring the Effect on Neuroinflammation of a Composition (a) According to the Invention Comprising a Mixture of Molecules of Natural Origin

In this example, composition (a) according to the invention is called “A.P.I” for “Active Propolis Ingredients”.

### Objective:

The aim of this example is to study the effect of composition (a) according to the invention comprising a mixture of molecules of natural origin, characterized in that said mixture is a propolis extract, on the response of human microglial cells following their activation by lipopolysaccharide (LPS) by quantifying the secretion of pro-inflammatory and anti-inflammatory cytokines in vitro.

### Methods:

Cell culture for microglial cell proliferation was performed according to the cell supplier's instructions. The culture for microglial cell differentiation and stimulation with LPS (lipopolysaccharide from Escherichia coli 055:B5) were performed as previously described (Ohgidani et al, Scientific Reports, 2014).

Briefly, 1.5 million cryopreserved primary human brain microglial cells were thawed and resuspended in a proliferation medium consisting of RPMI1640 cell culture medium supplemented with 10% heat-inactivated fetal bovine serum, 10 ng/mL M-CSF and 1% antibiotics/antimycotics.

Cells were grown for 15 days in a 175 cm² flask to a minimum of 9 million cells, then divided onto a 96-well multiwell microplate and grown for 4 days in serum-free RPMI-1640 cell culture medium supplemented with 10 ng/mL M-CSF and 100 ng/mL IL-34 under standard culture conditions (37° C., 5% CO2).

In this culture medium, cell numbers remain unchanged over 4 days. Results are therefore normalized to the number of cells.

After 4 days' incubation, the test compounds were applied 1 hour before treatment with LPS (0.5 μg/mL, for 6 hours). After applying PS, cell culture supernatants were collected and stored at −80° C. for further processing.

The compounds and their respective concentrations are described below:

TABLE 4

| | Composition (a) according to the invention | | | |
|---|---|---|---|---|
| | (A.P.I) (μg total polyphenols/mL) | | | |
| | 6 | 12 | 24 | 48 |
| PINOCEMBRIN (μM) | 1.23 | 2.47 | 4.93 | 9.86 |
| CHRYSIN (μM) | 1.08 | 2.15 | 4.31 | 8.62 |
| CAPE (μM) | 0.44 | 0.88 | 1.76 | 3.53 |

### Protocol:

Three multi-well 96 plates were used
Plate 1: 12-well microplate at a density of 2×10⁴ cells/mL used to validate the protocol
Plate 2: 96-well microplate at a density of 2×10⁴ cells/mL for studying the effect of the extract according to the invention (A.P.I.) on TNF-alpha, IL-6, IL10, IL-1beta, IFN-gamma
Plate 3: 96-well microplate at a density of 5×10⁴ cells/mL to study the effect of the extract according to the invention (A.P.I) on secretion of various cytokines TNF-alpha, IL-6, IL-10, IL-1beta, IFN-gamma by human microgial cells The positive control used in this experiment is Ibuprofen 200 μM.

The cultures were performed as follows:
Step 1: 15-day culture of microglia in proliferation medium to a minimum number of cells for seeding the three plates
Step 2: Plating three plates
Step 3: 4 days in a differentiation medium
Step 4: Stimulation of plate 1 with LPS and collection of supernatant 6 hours after stimulation
Step 5: Measurement of TNF Alpha in plate 1 by ELISA and validation of the protocol

A significant increase in TNF-alpha levels >2 times the levels observed in control cultures is expected in LPS-treated cultures (for at least one of the 2 concentrations).
Step 6: Stimulation of Plate 2 and Plate 3 with LPS 1 hour after treatment and collection of the supernatant 6 hours after stimulation using the protocol validated for Plate 1
Step 8: Measurement of cytokines in the supernatant with multiplex assay

### Conclusion:

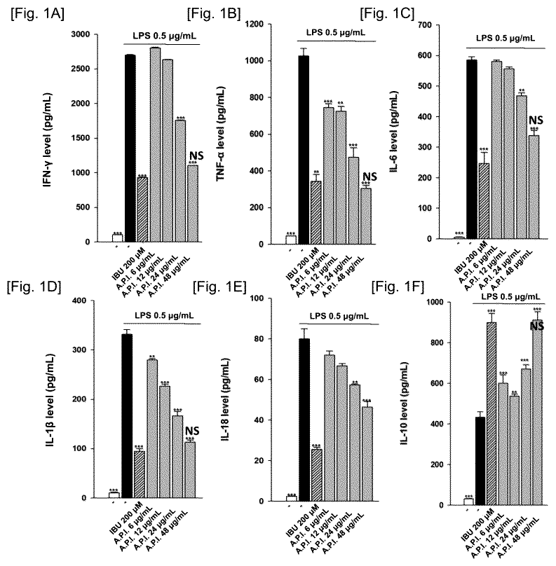

The production of pro-inflammatory cytokines is described in FIG. 1 a) INF-gamma, b) TNF-alpha, c) IL-6, d) IL-1beta, e) IL-18) and anti-inflammatory f) IL-10) expressed in pg/mL, was measured by ELISA assays (Multplex plate, R&D systems, ref Quantikine) following LPS stimulation (0.5 μg/mL, 6 hours). *** p<0.001, ** p<0.01 one-way ANOVA vs LPS, followed by Student-Newman-Keuls Method test. NS=Not significant compared with IBU (Ibuprofen, positive control). Neurons were pretreated for 1 hour with LPS neurotoxin using composition (a) according to the invention (A.P.1).

It can be seen that composition (a) according to the invention (A.P.I) at a concentration of 48 μg PPTx/mL has an effect significantly no different from that of ibuprofen on the expression of IFN gamma, TNF-alpha, IL-6, IL1-beta. Consequently, it is observed that composition (a) according to the invention comprising a mixture of molecules of natural origin, characterized in that said mixture is a propolis extract comprising chrysin, apigenin and CAPE possesses an anti-inflammatory activity similar to that of ibuprofen on the inhibition of the secretion of CNS pro-inflammatory cytokines.

FIG. 1 F) also shows that the activity of composition (a) according to the invention (A.P.I) and ibuprofen is similar on the expression of IL-10, an anti-inflammatory cytokine.

### Example 8: Effect of a Composition (a) According to the Invention Comprising a Mixture of Molecules of Synthetic Origin on Neuroinflammation

The effect on neuroinflammation was measured under the same conditions as in Example 7.

Pinocembrin, chrysin and CAPE obtained by chemical synthesis were tested individually and then in combination to demonstrate synergy on the expression of pro-inflammatory cytokines involved in neuroinflammation.

### Conclusion:

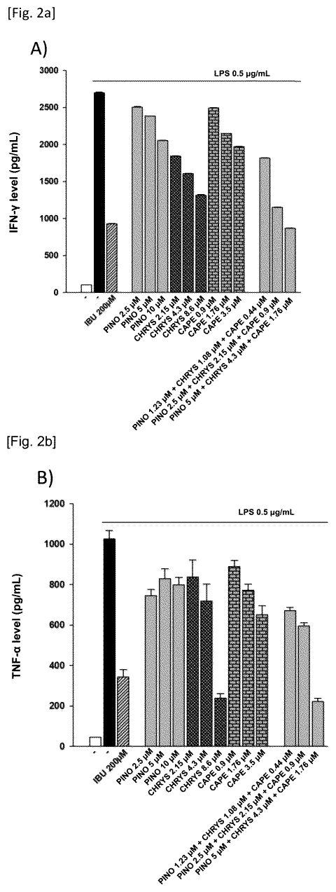

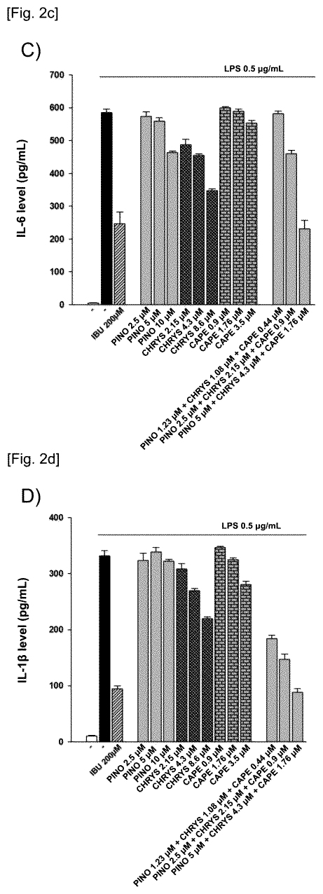

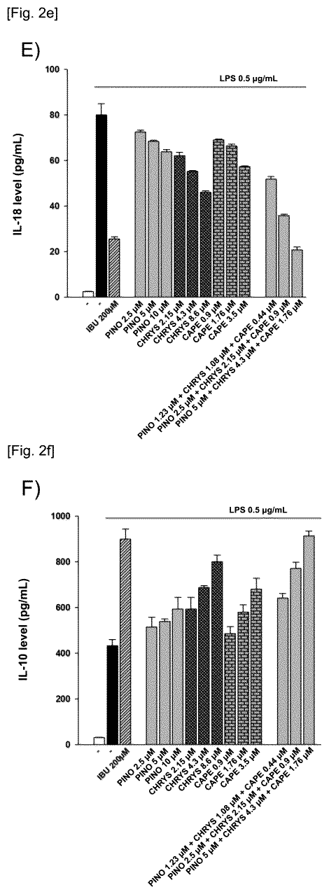

Synergy between chrysin, pinocembrin and CAPE was observed in the production of pro-inflammatory cytokines (results shown in FIG. 2a) INF-gamma, b) TNF-alpha, c) IL-6, d) IL-1 beta, e) IL-18) and anti-inflammatory cytokines f) IL-10) expressed in pg/mL. Cytokine production was measured by ELISA assays (Multplex plate, R&D systems, ref Quantikine) following LPS stimulation (0.5 μg/mL, 6 hours). *** p<0.001, ** p<0.01 one-way ANOVA vs LPS, followed by Student-Newman-Keuls Method test.

A more pronounced synergistic effect is observed when composition (a) of the mixture comprises molecules at the following concentration:
5 μM pinocembrin;
4.3 μM chrysin; and
1.76 μM CAPE.

As a result, the inventors have demonstrated a synergistic effect of the combination of chrysin, CAPE and pinocembrin on neuroinflammation.

### Example 9: Study of the Anxiolytic Effect of Composition (a) According to the Invention Comprising Propolis Extract in a Behavioral Anxiety Test (Elevated Plus Maze Test)

In this example, composition (a) according to the invention comprising a propolis extract is called “A.P.I”, for “Active Propolis Ingredient”.

Male BalbC mice, which have an anxiogenic genetic background, (10-14 weeks old, weight 25-30 i) were administered 30 minutes prior to the elevated plus maze anxiety behavioral test, divided into:
Group 1 (Composition (a) according to the invention (A.P.I); dose 451 mg total polyphenols/kg body weight oral gavage, n=16 mice)
Group 2 (ctl>0; DIAZEPAM 1 mg/Kg, n=16 mice),
Group 3 (ctl<0; NaCl 0.9% (n=16 mice)).

Statistical tests p<0.01, **, compared with group <0 (NaCl 0.9%).

The compounds and their respective concentrations are described below:

TABLE 5

| Composition (a) according | |
|---|---|
| to the invention (A.P.I) | Dilution ¼ of composition |
| administered per animal | (a) according to the invention |
| ″A.P.I. volume (μL) | 37.5 |
| NaCl 0.9% q.s.p 150 (μL) | 122.5 |
| Oral force-feeding/mouse (μL) | 150 |
| PPTx (total polyphenols) | 451 mg/kg |
| PINOCEMBRIN (mg/Kg) | 23.7 |
| CHRYSIN (mg/Kg) | 20.5 |
| APIGENIN (mg/Kg) | 2.2 |

Behavioral anxiety is measured for 5 minutes in the elevated plus maze test, the standard test for assessing anxiety in rodents. The structure is formed by two face-to-face open arms and two face-to-face closed arms. The whole structure forms a cross raised off the ground. This test is based on rodents' natural fear of open spaces and heights. Thus, the more anxious an animal is, the more it restricts itself to the closed arms.

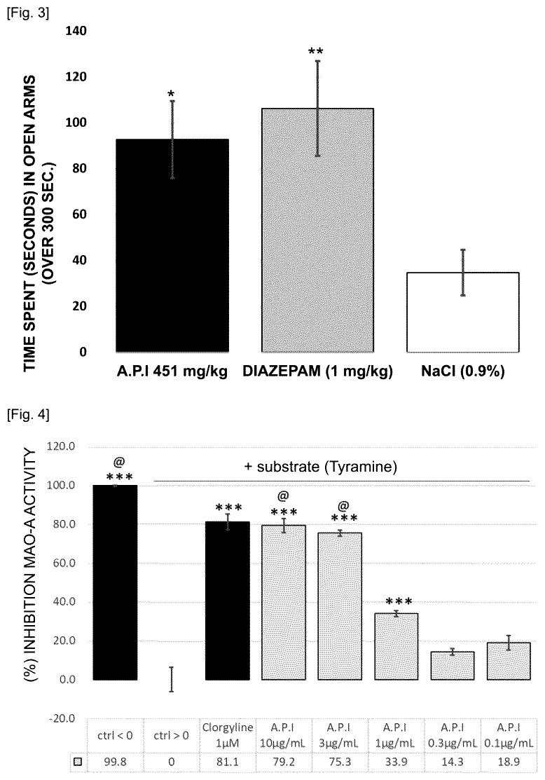

FIG. 3 shows the anxiolytic effect of composition (a) according to the invention. It can be seen that composition (a) according to the invention has a significant effect compared with the control and similar to that of DIAZEPAM, the reference molecule for combating anxiety.

### Example 10: Study of the Inhibitory Effect of Composition (b) According to the Invention Comprising a Propolis Extract on the Enzymatic Activity of Monoamine Oxidase A—Depression

The dose-response inhibitory effect of the extract according to the invention on the enzymatic activity of Monoamine oxidase type A (MAO-A) was carried out using an ELISA assay measuring MAO-A enzymatic activity.

The experimental conditions are as follows:
presence of substrate (control>0)
absence of substrate
presence of MAO-A substrate+specific inhibitor of MAO-A activity (clorgyline)

$$mAO - A substrate + composition ( b ) according to invention ( A . P . I ) - 0.1 - 10 µg / mL .$$

Experimental conditions over 20 minutes. 96 wells, 16 different conditions, n=5/condition.

Composition (b) according to the invention comprises a propolis extract whose composition is described in the table below.

TABLE 6

| | Composition (b) according to the invention | | | | |
|---|---|---|---|---|---|
| | (A.P.I) μg total polyphenols/mL | | | | |
| | 0.1 | 0.3 | 1.0 | 3.0 | 10.0 |
| GALANGIN (μM) | 0.0141 | 0.0422 | 0.1405 | 0.4216 | 1.4053 |
| APIGENIN (μM) | 0.0018 | 0.0055 | 0.0182 | 0.0547 | 0.1823 |

The results of this study are shown in FIG. 4.

### Statistical Analyses:

Multiple comparisons were performed according to Tukey's test), *** P<0.001 vs. control >0 (no inhibitor), @indicates no statistical difference vs. inhibitor control (clorgyline).

### Conclusion:

FIG. 4 shows that composition (b) according to the invention comprising a mixture of molecules of natural origin, characterized in that said mixture is a propolis extract, has a similar effect to Clorgyline on the inhibition of monoamine oxidase A activity at concentrations of 3 μg/mL and 10 μg/mL. Clorgyline is a standard molecule for inhibiting monoamine oxidase A. As a result, this example clearly demonstrates the synergistic effect of composition (b) according to the invention on depression.

### Example 11: Study of the Inhibitory Effect of Composition (b) According to the Invention Comprising a Mixture of Molecules of Synthetic Origin on the Enzymatic Activity of Monoamine Oxidase A—Depression

The inhibitory effect on the enzymatic activity of monoamine oxidase Awas studied under the same conditions as in Example 10.

Chemically synthesized galangin and apigenin were tested individually and then in combination to demonstrate synergy in inhibiting monoamine oxidase A enzymatic activity.

### Conclusion:

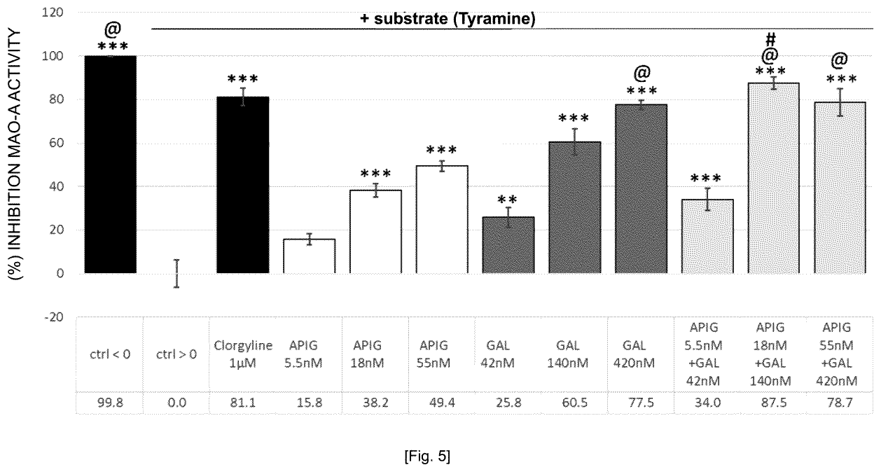

FIG. 5 shows the synergy between apigenin and galangin on monoamine oxidase A activity.

A more pronounced synergistic effect is observed when composition (b) comprises a mixture of molecules comprising:
18 nM apigenin; and
140 nM galangin.

The inventors have thus demonstrated a synergistic effect of the combination of galangin and apigenin on the enzymatic activity of monoamine oxidase A and consequently an effect on depression.

### Example 12: Study of the Inhibitory Effect of Composition (b) According to the Invention Comprising a Propolis Extract on the Enzymatic Activity of Monoamine Oxidase B—Parkinson's Disease

The dose-response inhibitory effect of composition (b) according to the invention comprising a propolis extract on the enzymatic activity of monoamine oxidase B (MAO-B) was carried out using an ELISA assay

The experimental conditions are as follows:
presence of substrate (control >0)
absence of substrate
presence of MAO-B substrate+specific inhibitor of MAO-B activity (selegiline)
MAO-B substrate+extract according to the invention (A.P.I)—0.1-100 μg/mL.

Experimental conditions over 20 minutes. 96 wells, 16 different conditions, n=5/condition.

The composition of the extract according to the invention used in this example is described in the table below.

TABLE 7

| | Composition (b) according to the invention | | | | |
|---|---|---|---|---|---|
| | (A.P.I) μg total polyphenols/mL | | | | |
| | 0.1 | 0.3 | 1.0 | 30 | 100 |
| GALANGIN (μM) | 0.0141 | 0.0422 | 0.1405 | 4.216 | 14.053 |
| APIGENIN (μM) | 0.0018 | 0.0055 | 0.0182 | 0.547 | 1.823 |

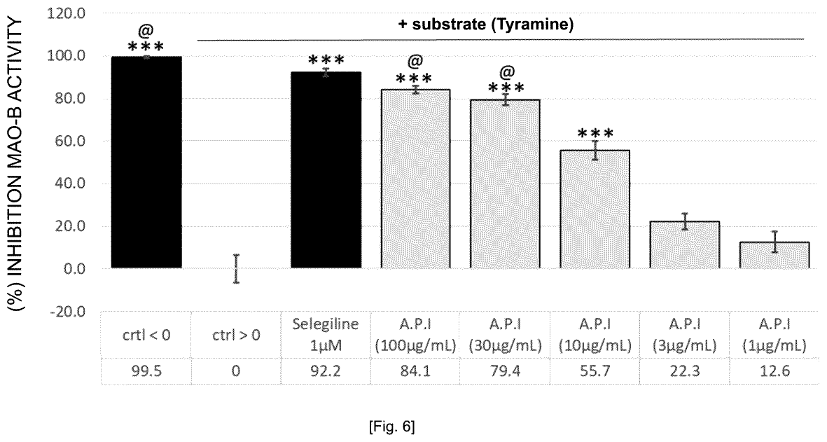

The results of this study are shown in FIG. 6.

### Statistical Analyses:

Multiple comparisons were performed according to Tukey's test), *** P<0.001 vs. control >0 (no inhibitor), @indicates no statistical difference vs. inhibitor control (selegiline).

### Conclusion:

FIG. 6 shows that composition (b) according to the invention comprising a mixture of molecules of natural origin, characterized in that said mixture is a propolis extract, has a similar effect to selegiline on the inhibition of monoamine oxidase B activity at concentrations of 10 μg/mL, 30 μg/mL, and 100 μg/mL. Selegiline is a standard molecule for inhibiting monoamine oxidase B activity.

Consequently, this example clearly demonstrates the effect of composition (b) according to the invention on the inhibition of monoamine oxidase B and consequently on Parkinson's disease.

### Example 13: Study of the Inhibitory Effect of Composition (b) According to the Invention Comprising Mixture of Molecules of Synthetic Origin on the Enzymatic Activity of Monoamine Oxidase B—Parkinson's Disease

The measurement of the inhibitory effect on the enzymatic activity of monoamine oxidase B was studied under the same conditions as in Example 12.

Chemically-synthesized galangin and apigenin were tested individually and then in combination to demonstrate synergy in inhibiting monoamine oxidase B enzymatic activity.

### Conclusion:

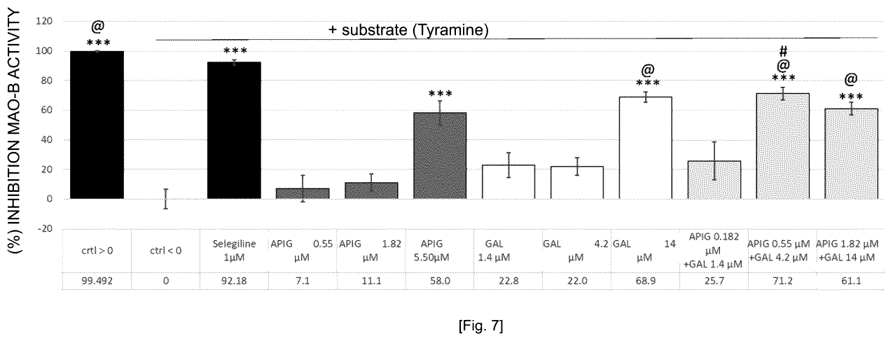

The results presented in FIG. 7 clearly show a synergy between apigenin and galangin on monoamine oxidase type B activity.

A more pronounced synergistic effect is observed when composition (b) comprises a mixture of molecules comprising:
0.55 μM apigenin; and
4.2 μM galangin.

The inventors have thus demonstrated a synergistic effect of the combination of galangin and apigenin on the enzymatic activity of monoamine oxidase B and consequently an effect on Parkinson's disease.

### Example 14: Study of the Inhibitory Effect of Composition (c) According to the Invention Comprising a Propolis Extract on the Enzymatic Activity of Acetylcholinesterase—Alzheimer's Disease/Cognitive Decline/Cerebral Aging

The effects of composition (c) according to the invention (A.P.I) comprising a mixture of molecules of natural origin, characterized in that the said mixture is a propolis extract, on the enzymatic activity of acetylcholinesterase were evaluated using an ELISA assay.

These products represent a calibrated kit with optimized concentrations for each component of the kit (enzymes, substrate, inhibitor control), enabling acetylcholinesterase activity and the inhibitory properties of the various compounds tested to be assessed under optimal conditions.

This screening kit for acetylcholinesterase inhibitors is based on an improved Ellman method, wherein thiocholine produced by the action of acetylcholinesterase forms a yellow color with 5,5-dithiobis (2-nitrobenzoic) acid (DTNB).

The intensity of the product color, measured at 412 nm, is proportional to the enzymatic activity of the sample.

Thus, this kit comprises a sample dilution buffer, substrate (acetylcholine) and DTNB. Acetylcholinesterase and Donepezil positive control are supplied separately.

Donepezil is a standard treatment for Alzheimer's disease.

To study the effect of composition (c) according to the invention, the test was adapted for 96-well plates. A fixed concentration of substrate (acetylcholine) and variable concentrations of composition (c) according to the invention were used (1 μg/mL −100 μg total polyphenols/mL).

Samples, substrate and enzyme were prepared and diluted precisely according to the kit manufacturer's instructions.

The assay was carried out with the addition of inhibitor. Inhibition was calculated as the percentage of product formation relative to the corresponding control (enzyme-substrate reaction) without inhibitors.

Reactions were carried out in the buffer solution from the kit supplied by the manufacturer. Incubation mixtures contained 45 μL of enzyme solution, 150 μL of substrate solution and 5 μL of test compound solution (or control or inhibitor control) for a total of 200 μL.

Test compounds were dissolved in DMSO to give a final concentration of 1% DMSO in 5 μL. The total reaction volume was 200 μL, so the final concentration of DMSO in the reaction mixture was 0.05%.

The enzyme solution and test compound solution mixtures were preincubated for 15 min at 25° C. The substrate solution was then added to the mixture to initiate the reactions. The reaction mixture was then immediately placed in the plate reader for a first reading at T1=0 min and a second reading at T2=10 min. Acetylcholine is metabolized by the Enzyme in the reaction mixture, leading to the formation of yellow TNB (5-thio-2-nitrobenzoic acid).

This release is therefore correlated with enzyme activity. The test is therefore based on absorbance detection kinetics (412 nm).

Appropriate controls with/without the test compounds, composition (c) according to the invention (A.P.1), with/without enzyme or with/without substrate were configured and performed simultaneously to check for interference with fluorescence measurements.

Experimental conditions with 5 samples per condition:
a) substrate (control >0),
b) Presence of substrate+specific inhibitor of AChE activity (Donepezil 20 μM)
c) acetylcholine substrate+extract according to invention (A.P.I) 1-100 μg/mL.

Statistical analyses: Multiple comparisons posthoc (Tukey test), *** P<0.001 versus substrate alone

Composition (c) according to the invention comprises a propolis extract whose composition is described in the table below.

TABLE 8

| | Composition (c) according to the invention | | | | |
|---|---|---|---|---|---|
| | (A.P.I) μg total polyphenols/mL | | | | |
| | 1 | 3 | 10 | 30 | 100 |
| GALANGIN (μM) | 0.141 | 0.422 | 1.41 | 4.216 | 14.053 |
| CAPE (μM) | 0.07 | 0.022 | 0.73 | 2.2 | 7.35 |

### Conclusion:

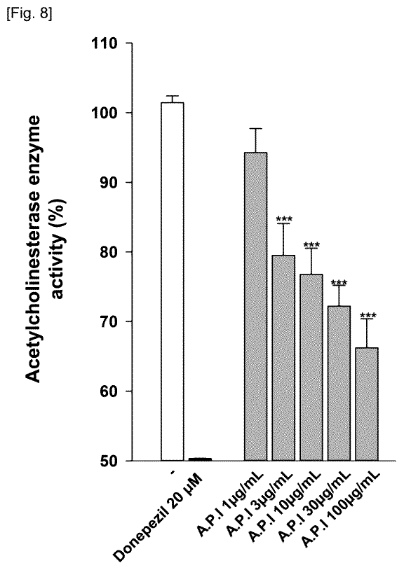

The results are shown in FIG. 8. These results show that composition (c) according to the invention comprising a mixture of molecules of natural origin, characterized in that said mixture is a propolis extract, has a similar effect to Donepezil on inhibition of acetylcholinesterase activity, at concentrations >30 μg/mL. Donepezil is a reference molecule for inhibiting acetylcholinesterase activity.

Consequently, this example clearly demonstrates the effect of composition (c) according to the invention on Alzheimer's disease and related disorders.

### Example 15: Study of the Inhibitory Effect of Composition (c) According to the Invention Comprising a Mixture of Molecules of Synthetic Origin on the Enzymatic Activity of Acetylcholinesterase—Alzheimer's Disease/Cognitive Decline/Cerebral Ageing

The results described in this example were obtained under the same experimental conditions as in Example 14.

Chemically synthesized galangin and CAPE were tested individually and then in combination to demonstrate synergy in inhibiting acetylcholinesterase activity.

### Conclusion:

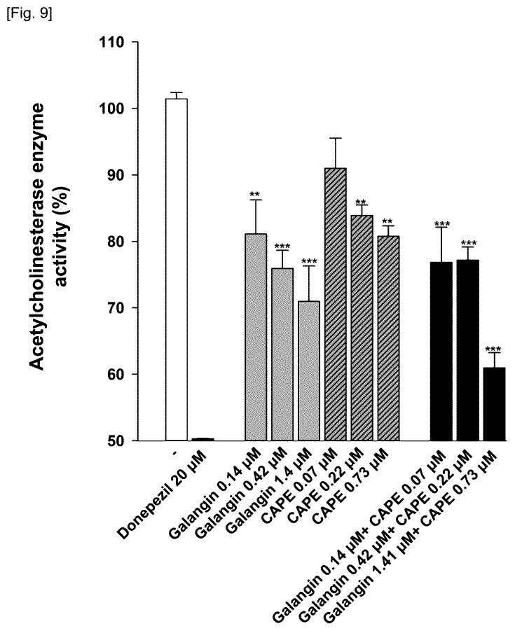

The results shown in FIG. 9 clearly demonstrate the synergy between galangin and CAPE on acetylcholinesterase activity.

In particular, a pronounced synergistic effect is observed when composition (c) comprises a mixture of molecules comprising:
20-1.41 μM galangin; and
0.73 μM CAPE.

The inventors have thus demonstrated a synergistic effect of the combination of galangin and CAPE on the enzymatic activity of acetylcholinesterase and consequently an effect on Alzheimer's disease and memory.

### Example 16: Study of the Neuroprotective Effect of Composition (a) According to the Invention Comprising a Propolis Extract

The aim of this study is to determine whether composition (a) according to the invention comprising propolis extract protects dopaminergic neurons against degeneration induced by 1-methyl-4-phenylpyridinium ion (MPP+) or 6-hydroxydopamine (6-OHDA).

### Methods:

### 1) Primary Culture of Mesencephalic Dopaminergic Neurons

Animals were treated in accordance with the Guide for the Care and Use of Laboratory Animals (National Research Council, 1996), European Directive 86/609, and the guidelines of the local institutional animal care and use committee. Cultures are prepared from the ventral mesencephalon of 15.5-day gestation Wistar rat embryos (Centre d'elevage Janvier, Le Genest St Isle, France). Dissociated suspension cells obtained by mechanical trituration of pieces of mesencephalic tissue are seeded at a density of 1.2-1.5 105 cells/cm2 on tissue culture media pre-coated with 1 mg/mL polyethylenimine diluted in borate buffer pH 8.3 as previously described (Michel et al., 1997). Cultures were maintained in Neurobasal medium supplemented with B27 (no antioxidant), 2 mM L-glutamine, 100 mg/mL streptomycin and 100 U/mL penicillin, as well as K+ (30 mM) and MK-801 (5 μM) to avoid excitotoxicity. The environment remained unchanged throughout the experiment. Note that tyrosine hydroxylase (TH+) neurons represent around 1-2% of the total number of neuronal cells present in these cultures.

### 2) Treatments

Cell culture treatments with A.P.I. were initiated 2 hours before application of MPP+ or 6-OHDA. MPP+ or 6-OHDA treatments will be initiated from in vitro day 7 to in vitro day 9. The vehicle used is the culture medium.

### Measurement and Analysis

### 1) Immunostaining

48 hours after MPP+ or 6-OHDA treatment, the cultures are fixed for 12 min with 4% formaldehyde in Dulbecco's phosphate-buffered saline (PBS), then washed twice with PBS before an incubation step at 4° C. for 24-48 hrs with the following antibody:
An anti-TH monoclonal antibody produced in mice diluted 1:5000 (anticorpsenligne, ref ABIN617906) to assess the number of DA neurons.

All antibodies are diluted in PBS containing 0.2% Triton X-100. Primary antibody detection is carried out with a CF-488 anti-mouse secondary antibody (TH; 1/500, Ozyme, ref BTM20208).

### Counting the Number of Cells

TH+ neurons are counted at ×200 using a ×20 objective coupled to a ×10 eyepiece. The number of TH+ neurons in each culture well is estimated after counting 5 visual fields distributed along the x and y axes.

### Statistical Analysis

The data are expressed as a percentage of control conditions (no treatment=100%). All values are expressed as mean+/−SEM (s.e.mean) (n=5 wells per condition per culture). Statistical analyses of the different conditions (ANOVA followed by Dunnett's test where permitted) are carried out using SigmaStat.

### Conclusion:

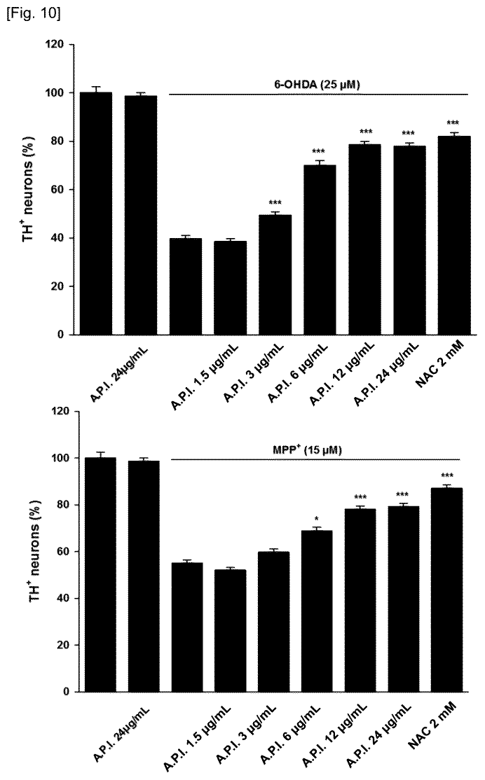

FIG. 10 shows a significant dose-dependent neuroprotective effect of composition (a) according to the invention comprising a mixture of natural molecules, characterized in that said mixture is a propolis extract.

### Example 17: Pharmacological Evaluation of the Anxiolytic and Sedative Behavioral Effects of Composition (a) According to the Invention in the Elevated Plus Maze (EPM) and Open-Field (OF) Tests in Mice

### Materials and Methods:

### Animals:

Male BALB/c mice arrived 3 weeks before the start of the experiments and were between 2 and 3 months old. On arrival, the mice were maintained under standard laboratory conditions: 4 to 5 mice per cage, 12 h-light/dark cycle, 22° C.+/−2, less than 40% humidity, and free access to food and water.

### Procedure:

propolis group with ¼ dilution (Composition (a) according to the invention as described in Example 9, 451 mg/kg, orally, 16 mice);
propolis group with ¼ dilution (Composition (a) according to the invention as described in Example 9, 451 mg/kg, orally) pretreated with flumazenil (2.5 mg/kg, intraperitoneally, 16 mice)
positive control group, diazepam (2 mg/kg, orally, 16 mice)
control group, vehicle solution alone (16 mice)

The study consisted in carrying out the 2 behavioral tests, as well as recovering a blood sample at the end of the tests to extract the serum for subsequent analysis, as described in example 18 below. During the procedure, each mouse received 2 injections spaced 15 minutes apart, corresponding (1) for the first, to pre-treatment administered intraperitoneally (ip) either with flumazenil (2.5 mg/kg, for 1 group) or with the vehicle solution (NaCl 0.9%, 3 groups), (2) for the second treatment administered orally (oral force-feeding) either with composition (a) according to the invention (¼ dilution, corresponding to 451 mg/kg, 2 groups), or with diazepam (2 mg/kg, 1 group), or with the vehicle solution (water/propylene glycol 25/75, 1 group).

Thirty minutes after the second injection, the tests were carried out: first the EPM, then the OF.

### The Elevated Plus Maze (EPM):

The device consisted of a raised Plexiglas maze (40 cm high) in the shape of a cross, with two opposite arms open to the void (without walls) and two opposite arms closed (the arms are 27 cm long and 5 cm wide). Each mouse was placed on the central square at the crossroads of the 4 arms at the start of the test. Its exploration behavior was recorded over a 5-minute period, including distance traveled and time spent in open arms.

### The Open-Field (OF) Test

The device consisted of a circular arena 35 cm in diameter with a wall 30 cm high. The arena was separated into 2 zones of interest by dividing the diameter in 2: a central circular zone with a diameter of 17.5 cm and a peripheral zone corresponding to the 17.5 cm-thick ring separating the central zone from the wall. Each mouse was initially placed in the peripheral zone, its head facing the wall, and then allowed to explore the arena freely for 30 minutes. Its exploration behavior was recorded over a 30-minute period, including distance traveled and time spent in the central zone.

### Statistics:

Results were analyzed using a one-factor ANOVA (“group” factor). If a significant difference was revealed by ANOVA (significance level 0.05), then a planned multiple comparison test using the Benjamini-Hochberg method (“False Discovery Rate”, FDR) was performed to determine which group(s) was (were) significantly different from the control group.

### Results:

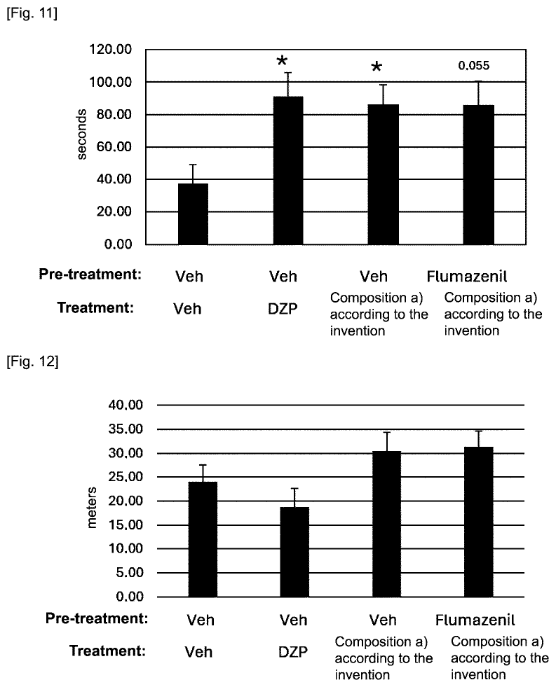

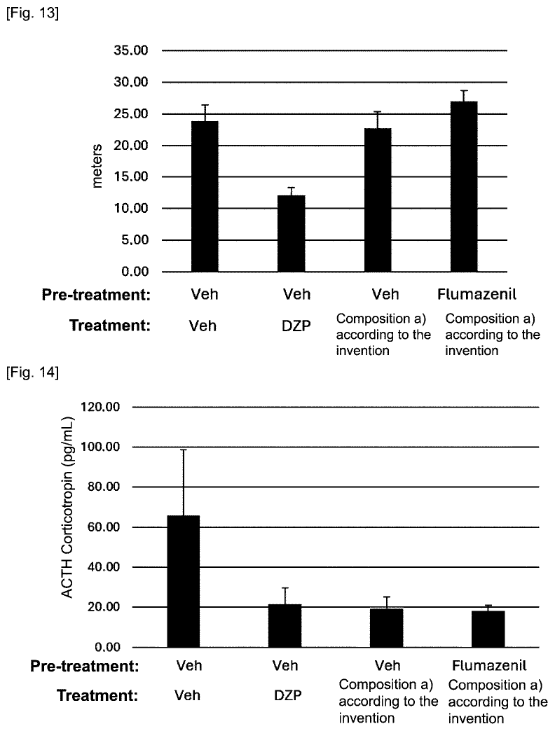

Behavioral results showed that composition (a) according to the invention diluted by ¼ was capable of inducing an anxiolytic-like effect in the elevated plus maze (EPM) test (FIGS. 11 and 12) as well as in the OF test (FIG. 13) with an effect size similar to that of a benzodiazepine, diazepam (2 mg/kg), the reference anxiolytic molecule.

Moreover, this effect was not accompanied by a sedative effect as in the case of diazepam, suggesting that composition (a) according to the invention does not induce the side effects described and observed with the reference molecule. Finally, the anxiolytic effect of composition (a) according to the invention was not antagonized by flumazenil, indicating that propolis extract can mobilize pathways other than that of GABA-A receptors alone to induce its anxiolytic effects.

This example deliberately does not show the anxiolytic effect observed after pretreatment with flumazenil and subsequent treatment with diazepam. This is because it has been widely demonstrated in the literature that the effect observed by diazepam was solely due to GABA-A receptors. Consequently, such an experiment would have demonstrated the absence of any effect of diazepam during pretreatment with flumazenil.

As EPM is considered the standard test for preclinical evaluation of anxiolytic effects, this is a positive result for the anxiolytic action of composition (a) according to the invention. Furthermore, the results show that the size of this effect is similar to that induced by the study's reference anxiolytic, diazepam.

On the other hand, while it has already been shown that benzodiazepines induce their anxiolytic behavioral effects via their action on the GABAA receptor pathway, this pathway is certainly not unique, nor is it necessary for the behavioral effects of composition (a) according to the invention, given that the latter was able to maintain comparable anxiolytic effects even following pretreatment with a GABA-A receptor antagonist, flumazenil.

This can be explained by the fact that composition (a) according to the invention is composed of several types of polyphenols with potential pharmacological properties on several elements of signaling or metabolic pathways of neurotransmitter systems, neuroplasticity and neuroinflammation.

These results demonstrate that the anxiolytic effects of composition (a) according to the invention are based on a pleiotropic pharmacological action of its various constituents, contributing to a mitigation of anxiety in light of its composition.

### Example 18: Impact of Taking Composition (a) According to the Invention on the Production of Stress Hormones (ACTH and Corticosterone), Following Behavioral Tests

### Materials and Methods:

At the end of the OF described in Example 17, blood was immediately drawn via the mandibular vein prior to euthanasia. It was then centrifuged at ×1000 g for 15 minutes and the plasma was collected as supernatant and stored at −80° C. ELISA assays were performed on the recovered plasma (approx. 100 μL) to measure the stress hormones ACTH and Corticosterone.

### Procedure:

propolis group with ¼ dilution (Composition (a) according to the invention as described in Example 9, 451 mg/kg, orally, 6 mice);
propolis group with ¼ dilution (Composition (a) according to the invention as described in Example 9, 451 mg/kg, orally) pretreated with flumazenil (2.5 mg/kg, intraperitoneally, 8 mice)
positive control group, diazepam (2 mg/kg, orally, 7 mice)
control group, vehicle solution alone (8 mice).

The results shown in FIG. 14 demonstrate that taking composition (a) according to the invention induces a reduction in ACTH concentration in the blood similar to the action of diazepam, the reference molecule.

1. A composition (a) comprising a mixture of molecules comprising pinocembrin, chrysin and caffeic acid phenethyl ester (CAPE), for use in the prevention or treatment of neuroinflammation and/or diseases or disorders associated with neuroinflammation and/or diseases or disorders associated with neuroinflammation.

2. The composition (a) for its use according to claim 1, characterized in that composition (a) is liquid and contains no alcohol.

3. The composition (a) for its use according to claim 1, characterized in that composition (a) further comprises at least one additive.

4. The composition (a) for its use according to claim 1, characterized in that the mixture of molecules comprises:
at least 5 μM pinocembrin;
at least 4.3 μM chrysin; and
at least 1.76 μM CAPE.

5. The composition (a) for its use according to claim 1, characterized in that the mixture of molecules further comprises at least one molecule chosen from apigenin, galangin and combinations thereof.

6. The composition (a) for its use according to claim 1, characterized in that the mixture of molecules is of natural origin.

7. The composition (a) for its use according to claim 1, characterized in that the mixture of molecules is an extract of propolis.

8. The composition (a) for its use according to claim 7, characterized in that the propolis extract comprises, by weight of total polyphenols of the extract:
at least 2.6% CAPE
at least 8% pinocembrin; and
at least 8% chrysin.

9. The composition (a) for its use according to claim 7, characterized in that the propolis extract comprises, by weight of total polyphenols of the extract:
at least 0.4% apigenin; and/or
at least 6.0% galangin.

10. The composition (a) for its use according to claim 7, characterized in that the propolis extract comprises at least 70% of polyphenols in aglycone form.

11. The composition (a) for its use according to claim 7, characterized in that the propolis extract is obtained from at least one propolis selected from poplar propolis, Baccharis propolis and Dalbergia propolis and combinations thereof.

12. The composition (a) for its use according to claim 7, characterized in that the propolis extract has a viscosity of between 25 and 900 mPa·s measured at 20° C. using a Brookfield viscometer.

13. The composition (a) for its use according to claim 7, characterized in that the propolis extract is capable of being obtained by a method comprising a step of saponification.

14. The composition (a) for its use according to claim 7, characterized in that the propolis extract is capable of being obtained by a method comprising the implementation of the following steps:
a) Grinding at least one raw propolis,
b) Macerating the propolis powder obtained in step a) in an extraction solvent,
c) Filtering the macerate obtained in step b) and recovering the filtrate,
d) Evaporating the filtrate obtained in step c) forming a residue,
e) Mixing the residue obtained in step d) with a mixture of organic solvent and aqueous solvent with stirring,
f) Settling the mixture obtained in step e) and recovering the organic phase,
g) Washing the organic phase obtained in step f) with anhydrous sodium sulfate salts,
h) Filtering the organic phase obtained in step g),
i) Evaporating the organic solvent and recovering a liquid propolis extract comprising at least 18% polyphenols relative to the total mass of the extract.

15. The composition (a) for use according to claim 14, characterized in that the extraction solvent of step b) comprises an organic solvent, an aqueous solvent, and a strong base.

16. The composition (a) for use according to claim 15, characterized in that the strong base is present at a concentration of between 0.01 and 0.1 M.

17. The composition (a) for use according to claim 16, characterized in that the strong base is chosen from potassium hydroxide and sodium hydroxide.

18. The composition (a) for its use according to claim 14, characterized in that the propolis extract is obtainable by a method further comprising a step g) of washing the organic phase obtained in step f) with anhydrous sodium sulfate salts.

19. The composition (a) for its use according to claim 1, to prevent and/or combat a diseases and/or disorder associated with neuroinflammation selected from among Alzheimer's disease, Parkinson's disease, multiple system atrophy, Huntington's disease, posterior cortical atrophy, Pick's disease, epilepsy, vascular dementia, frontotemporal dementia, Lewy body dementia, amyotrophic lateral sclerosis, Korsakoff syndrome, alcohol withdrawal, ischemia, neonatal ischemia, head trauma, stroke, vascular dementia, cerebral ischemia, multiple system atrophy, progressive supranuclear palsy, amyotrophic lateral sclerosis, multiple sclerosis, narcolepsy, SARS-CoV-2, schizophrenia, bipolar disorder, anxiety disorders, depressive disorders, panic attacks, post-traumatic stress disorder, and autism.
